(19) United States
(12) Patent Application Publication
(10) Pub. No.: US 2026/0043024 A1
(43) Pub. Date: Feb. 12, 2026

(54) MIRNAS TARGETING ATNX2 FOR THE TREATMENT OF ALS AND SCA2

(71) Applicant: THE CHILDREN'S HOSPITAL OF PHILADELPHIA, Philadelphia, PA (US)

(72) Inventors: Beverly DAVIDSON, Philadelphia, PA (US); Defne A. AMADO, Philadelphia, PA (US); Alejandro Mas MONTEYS, Philadelphia, PA (US)

(73) Assignee: THE CHILDREN'S HOSPITAL OF PHILADELPHIA, Philadelphia, PA (US)

(21) Appl. No.: 19/102,997

(22) PCT Filed: Aug. 8, 2023
(86) PCT No.: PCT/US2023/071845

§ 371 Date: Feb. 11, 2025

Related U.S. Application Data
(60) Provisional application No. 63/373,637, filed on Aug. 26, 2022.

(51) Int. Cl. C12N 15/113; A61P 25/00; C12N 15/86

(52) U.S. Cl. CPC C12N 15/113; A61P 25/00; C12N 15/86; C12N 2310/141; C12N 2750/14143

(57) ABSTRACT

Provided herein are compositions and methods for treatment of Amyotrophic Lateral Sclerosis as well as Spinocerebellar Ataxia Type 2 by way of reducing levels of the ATXN2 gene expression. More specifically. miRNA compositions that target ATXN2 are able to reduce ATXN2 levels.

### PRIORITY CLAIM

This application claims benefit of priority to U.S. Provisional Application Ser. No. 63/373,637, filed Aug. 26, 2023, the entire contents of which are hereby incorporated by reference.

### STATEMENT REGARDING FEDERALLY FUNDING RESEARCH

This invention was made with government support under grant no. NS114106 awarded by The National Institutes of Health. The government has certain rights in the invention.

### BACKGROUND

### 1. Field

The present disclosure relates generally to the fields of neurology, medicine, and molecular biology. More particularly, it concerns miRNA compositions and methods of their use in treating Amyotrophic Lateral Sclerosis as well as Spinocerebellar Ataxia Type 2 by way of reducing expression levels of the ATXN2 gene.

### 2. Description of Related Art

Spinocerebellar ataxias (SCAs), also known as spinocerebellar atrophy or spinocerebellar degeneration, are progressive, degenerative, genetic diseases, each of which could be considered a neurological condition in its own right. An estimated 150,000 people in the United States have a diagnosis of spinocerebellar ataxia at any given time. SCA is hereditary, progressive, degenerative, and often fatal. There is no known effective treatment or cure. SCA can affect anyone of any age. The disease is caused by either a recessive or dominant gene. In many cases people are not aware that they carry a relevant gene until they have children who begin to show signs of having the disorder.

Amyotrophic lateral sclerosis (ALS) has the distinction of being both the most common and deadliest adult motor neuron diseases, with progressive loss of upper and lower motor neurons leading to paralysis and respiratory failure. A hindrance to disease-modifying therapy has been a lack of understanding of disease mechanism; indeed, out of >70 human clinical trials, only Riluzole and Edaravone have slowed progression, both minimally and by targeting non-specific factors such as excitotoxicity or oxidative stress. Recent advances in the understanding of ALS pathophysiology have laid the groundwork for targeted treatment, but true disease modifying therapy remains hindered by a lack of easily translatable approaches.

Spinocerebellar ataxia 2 (SCA2) is inherited in an autosomal dominant manner. This means that having one changed (mutated) copy of ATXN2 in each cell is enough to cause signs and symptoms of the condition. The ATXN2 gene mutations that cause SCA2 involve a DNA sequence called a ‘CAG trinucleotide repeat.’ It is made up of a series of three DNA building blocks (CAG stands for cytosine, adenine, and guanine) that appear multiple times in a row. The CAG sequence is normally repeated about 22 times in the gene, but it can be repeated up to 26 or 27 times. Repeats ranging from 26 to 27, 28, 29, 30, 31 or 32 are associated with the development of ALS. SCA2 develops in people who have 33 or more CAG repeats in the ATXN2 gene.

The ataxin-2 protein, which is encoded by ATXN2, is necessary for the formation of stress granules in the cytoplasm of neurons. Stress granules bind up many important proteins in cells but are usually transient and dissolve after the stressor is removed. Among these proteins is TDP-43, which normally is located in the cell nucleus. For reasons that are poorly understood, in ALS, TDP-43 progressively aggregates in stress granules without dissolving, ultimately leading to toxic cytoplasmic TDP-43 aggregates and a clearing of TDP-43 from the nucleus. This results in the death of motor neurons in the brain and spinal cord. It has been shown by others that inhibiting ATXN2 using antisense oligonucleotides partially inhibits this pathology and leads to prolongation of lifespan in an ALS mouse model with TDP-43 pathology.

A hallmark of ALS is cytoplasmic inclusions consisting largely of Tar-DNA binding protein of 43 kDA (TDP-43). Normally found in the nucleus, TDP-43 mediates splice regulation and transcriptional repression. Its cytoplasmic mislocalization is detrimental on two fronts: 1) toxicity of cytoplasmic aggregates, and 2) loss of normal nuclear function. In motor neurons from ALS patients, TDP-43 associates with cytoplasmic stress granules (SGs), conglomerates of proteins and RNA that become maladaptive in disease. SGs bind nuclear import and export factors, impairing shuttling and impeding transcription and translation. Intervening in this pathway could delay or prevent neuronal death.

Thus, improved therapies for SCAs and other ATXN2-related diseases are urgently needed.

### SUMMARY

Provided herein are adeno-associated viruses (AAV) encoding an miRNA selected from the group consisting of SEQ ID NO: 1, SEQ ID NO: 2, SEQ ID NO: 3, operably linked to a promoter. In some aspects, the miRNA is SEQ ID NO: 1. In some aspects, the miRNA is SEQ ID NO: 2. In some aspects, the miRNA is SEQ ID NO: 3. In some aspects, the miRNA is under the control of a first promoter, e.g., a U6 promoter. In some aspects, the modified AAV comprises capsid proteins derived from or selected from the group consisting of AAV1, AAV2, AAV3, AAV4, AAV5, AAV6, AAV7, AAV8, AAV9, AAV10, AAV11, AAV12, AAV-rh74, AAV-rh10, and AAV-218 VP1, VP2 and/or VP3 capsid proteins, or capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 VP1, VP2 and/or VP3 capsid proteins. In some aspects, the modified AAV comprises first and second AAV ITRs derived from, comprise or consist of an AAV1, AAV2, AAV3, AAV4, AAV5, AAV6, AAV7, AAV8, AAV9, AAV10, AAV11, AAV12, AAV-rh74, AAV-rh10 or AAV-218 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 ITR sequence.

Provided herein are pharmaceutical compositions comprising AAV of any one of the present embodiments and a pharmaceutically acceptable carrier.

Provided herein are isolated and purified nucleic acids comprising a sequence selected from the group consisting of SEQ ID NO: 1, SEQ ID NO: 2, or SEQ ID NO: 3, or a sequence having at least about 90% sequence identity therewith. In some aspects, the sequence is SEQ ID NO: 1 or a sequence having at least about 92%, 95%, 96%, 97%, 98% or 99% sequence identity therewith. In some aspects, the sequence is SEQ ID NO: 2 or a sequence having at least about 92%, 95%, 96%, 97%, 98% or 99% sequence identity therewith. In some aspects, the sequence is SEQ ID NO: 3 or a sequence having at least about 92%, 95%, 96%, 97%, 98% or 99% sequence identity therewith. In some aspects, the sequence is located in a replicable vector. In some aspects, the replicable vector is a non-viral vector. In some aspects, the replicable vector is a viral vector. In some aspects, the viral vector is an AAV vector. In some aspects, the miRNA is under the control of a first promoter, e.g., a U6 promoter. In some aspects, the modified AAV comprises capsid proteins derived from or selected from the group consisting of AAV1, AAV2, AAV3, AAV4, AAV5, AAV6, AAV7, AAV8, AAV9, AAV10, AAV11, AAV12, AAV-rh74, AAV-rh10, and AAV-218 VP1, VP2 and/or VP3 capsid proteins, or capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 VP1, VP2 and/or VP3 capsid proteins. In some aspects, the modified AAV comprises first and second AAV ITRs derived from, comprise or consist of an AAV1, AAV2, AAV3, AAV4, AAV5, AAV6, AAV7, AAV8, AAV9, AAV10, AAV11, AAV12, AAV-rh74, AAV-rh10 or AAV-218 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 ITR sequence.

Provided herein are methods of inhibiting expression of the gene encoding Ataxin 2 (ATXN2) comprising contacting a target cell with an miRNA selected from SEQ ID NO: 1, SEQ ID NO: 2, or SEQ ID NO: 3. In some aspects, the miRNA is naked miRNA. In some aspects, the miRNA is encapsulated in a nanoparticle (e.g., LNP) or liposome. In some aspects, the miRNA is encoding by an expression construct and expressed after uptake of the expression construct by the target cell. In some aspects, the expression construct is a non-viral construct. In some aspects, the expression construct is a viral construct, such as an AAV construct provided herein. In some aspects, the target cell is in a living subject. In some aspects, the administration to said living subject is systemic, regional, or localized, such as intravenous or directly to the central nervous system, including but not limited to delivery to the CSF, either via intracerebroventricular delivery, intra-cisterna magna delivery, or intrathecal delivery via lumbar puncture; intraparenchymal delivery, to the brain, brainstem, and/or spinal cord; or systemic. In some aspects, the miRNA is contacted or administered more than once, such as, for example, 2, 3, 4, 5, 6, 7, 8, 9, 10 or more times. In some aspects, the miRNA is contacted or administered monthly, every other month, every three months, every four months, every six months or annually. In some aspects, the methods further comprises providing an additional therapy to said subject. In some aspects, a plurality of viral particles, such as AAV vectors, are contacted or administered. In some aspects, the viral particles are administered to the living subject at a dose of about 1×10⁶ to about 1×10¹⁸ vector genomes per kilogram (vg/kg). In some aspects, the viral particles are administered to the living subject at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-1×10¹³, or about 1×10¹³-1×10¹⁴ vg/kg of the patient. In some aspects, the living subject is human. In some aspects, the living subject is a non-human mammal. In some aspects, the human subject has been diagnosed with SCA2 or ALS. In some aspects, the human subject has been determined to be at risk of SCA2 or ALS.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. Neonatal mice were injected in the bilateral lateral ventricles with AAV9 variants at post-natal day 0 (P0) or day 1 (P1) and sacrificed 4 weeks post-injection. Compared with controls (FIG. 1D), injection with AAV9 (FIG. 1A) showed moderate cortical and weak spinal cord transduction, both improved with the AAV-PHP·eB variant (FIG. 1B). Injection with AAV-1999 (FIG. 1C) showed strong transduction throughout both brain and spinal cord at both P0 and P1 injection times.

FIGS. 2A-B. Mice were treated at P1 with eGFP delivered via AAV9, AAV-PHP·eB, or the novel AAV9-variant AAV-1999. In cortex (FIG. 2A) and in anterior horn cells of the spinal cord (FIG. 2B), AAV-1999 results in strong transduction at both P0 and P1, while other serotypes transduce more weakly and with diminished efficacy at P1.

FIGS. 3A-D. Human and mouse Ataxin 2 mRNA levels are suppressed by AAV·miS12 in vitro. HEK293 cells (FIG. 3A, FIG. 3C) and murine N2A cells (FIG. 3B, FIG. 3D) were transfected with vectors delivering miS12, an RNAi against ATXN2/Atxn2. miS12·eGFP showed successful transduction by fluorescence microscopy compared to a scrambled non-targeting miRNA (pAAV·miCtrl) (FIG. 3A, FIG. 3B). Both pAAV·miS12·GFP and pAAV·miS12 demonstrated knockdown of human ATXN2 by ˜35% (FIG. 3C) and mouse Atxn2 by ˜75% (FIG. 3D) as measured by QPCR. N=3 wells/condition, 4 replicates/well. Data=mean+/−SEM of biological replicates. *p<0.05, ***p<0.0005, #p<0.0001, ##p<0.00005. Legends for FIGS. 3C-D are the same and are left-to-right corresponding to top-to-bottom.

FIG. 4. Ataxin 2 mRNA levels are suppressed by AAV·RNAi miS12 in vivo. SCA2 mice overexpressing human ATXN2 were injected in deep cerebellar nuclei with AAV1 delivering miATXN2 variants 7 or 12 (miS7 or miS12), and human ATXN2 and mouse Atxn2 mRNA levels measured in the cerebellum by QPCR 1 month after injection. miS12 suppressed both human and mouse ATXN2/Atxn2 compared to miCtrl. Results are presented as mean+/−SEM; ***p<0.005.

FIGS. 5A-E. AAV-PHP·eB·miS12 mediates strong cortical and spinal cord transduction and knockdown. Adult wild-type mice were injected systemically with AAV-PHP·eB·eGFP (FIG. 5A) or AAV-PHP·eB·miAtxn2 miS12 (FIGS. 5B-E), using a non-targeting miRNA as a control (AAV-PHP·eB·miCtrl) and normalizing to buffer-injected controls. Mice show strong expression of eGFP throughout the motor cortex and anterior horn cells of the spinal cord, as well as robust CNS knockdown of Atxn2 after administration of miS12. N=4/group for all studies.

FIGS. 6A-C. AAV-PHP·eB·miAtxn2 mediates strong cerebellar Purkinje cell transduction and knockdown. Adult wild-type mice were injected systemically with AAV-PHP·eB·eGFP (FIG. 6A) or AAV-PHP·eB·miAtxn2 miS12 (FIGS. 6B-C), using a non-specific control vector (AAV-PHP·eB·miCtrl) and normalizing to buffer-injected controls. Mice show strong expression of eGFP throughout the cerebellar Purkinje cells, as well as robust cerebellar knockdown of human ATXN2 (FIG. 6B) and mouse Atxn2 (FIG. 6C) after administration of miAtxn2 miS12. N=4/group.

FIGS. 7A-B. Knockdown of ATXN2/atxn2 using the original miATXN2 (miS12) was compared to that achieved by variants P1-P4 in cultured human HEK293T cells (FIG. 7A) and mouse N2A cells (FIG. 7B) using QPCR and normalized to ATXN2/Atxn2 levels in untransfected cells (Untransf). Variants P1 and P3 cause robust knockdown of both human and mouse ATXN2/atxn2 mRNA.

FIGS. 8A-B. A psiCHECK-2 RNAi luciferase assay was performed to compare activity and strand-loading of the guide (FIG. 8A) and passenger (FIG. 8B) strands of the miATXN2 sequences. Compared to the original miATXN2 (miS12), miATXN2-variant 1 (P1) and miATXN2-variant 3 (P3) show improved strand loading by the guide strand (FIG. 8A) and decreased strand loading by the passenger strand (FIG. 8B).

FIG. 9. The original miATXN2 (miS12) and the re-engineered variants (v1-v4, also referred to as P1-P4) were analyzed by the program siSPOTR for predicted potential off-targeting score (POTS) and number of predicted target genes. Variants v1 and v3 were chosen for subsequent experiments based on their reduced passenger-strand POTS score and off-targeting compared to miS12.

FIG. 10. miRNA Sequences.

FIGS. 11A-E. TAR^{4/4} pups were injected at postnatal day 1 with a non-ATXN2-targeting control miRNA (miCtrl), miATXN2-V1, or miATXN2-V3. Four weeks later, brain and spinal cords were harvested (FIG. 11A) and Atnx2 mRNA levels in the frontal cortex (FIG. 11B), brainstem (FIG. 11C), cortical (FIG. 11D) and lumbar spine (FIG. 11E) were measured by QPCR. miATXN2-V1 and miATXN2-V3 achieved strong Atxn2 knockdown throughout the central nervous system. N=4/group.

FIGS. 12A-E. Wild-type pups of the same genetic background as the TAR^{4/4} strain were injected at postnatal day 1 with buffer, a non-Atxn2-targeting control miRNA (miCtrl), miATXN2-V1, or miATXN2-V3 and were assessed for brain and spinal cord inflammation four weeks later (FIG. 12A). mRNA levels in the astrocytic marker GFAP (FIGS. 12B-C) and the microglial marker Iba1 (FIGS. 12D-E) were measured in the frontal cortex (FIGS. 12B and 12D), and lumbar spine (FIGS. 12C and 12E) by QPCR. There was no evidence of inflammation. N=4/group.

FIG. 13. Wild-type pups of the same background strain as 127Q mice (a model of SCA2 overexpressing mutant human ATXN2 in cerebellar Purkinje cells) were injected at P1 with buffer; a non-ATXN2 targeting control miRNA (miCtrl); or re-engineered miATXN2 variants 1 (V1) or 3 (V3). Four weeks later, Atxn2 mRNA levels were measured by QPCR. miATXN2-V1 and miATNX2-V3 achieved strong Atxn2 knockdown in cerebellum (91% and 61%, respectively). N=2=6/group.

FIGS. 14A-C. Gait impairment scores were calculated for TAR^{4/4} mice over time to establish a phenotype (FIG. 14A); scores were calculated by scoring degrees of abdominal droop, limping, foot angling, kyphosis, tremor, and clasping (FIG. 14B). TAR^{4/4} pups were then treated with either miATXN2-V1 or buffer and compared to wild-type littermates at postnatal day 18 (FIG. 14C). miATXN2-V1-treated mice showed marked reduction in gait impairment. N=12-38/group.

FIGS. 15A-E. Individual components of the gait impairment score were compared for miATXN2-V1-treated versus buffer-treated TAR^{4/4} mice at postnatal day 18. Treated mice showed marked improvement in limping (FIG. 15A), foot angling (FIG. 15B), kyphosis (FIG. 15C), tremor (FIG. 15D) and clasping (FIG. 15E). N=12-38/group.

FIG. 16. Latency to fall on rotarod was compared for miATXN2-V1-treated versus buffer-treated TAR^{4/4} mice at postnatal day 17. Treated mice showed 2.6× the rotarod endurance of untreated TAR^{4/4} mice. N=10-31/group.

FIG. 17. TDP-43^{+/+} pups were injected at P1 with buffer (VBS) or with miATNX2-V1. miATXN2-V1-treated mice showed a 45% increase in median lifespan compared to buffer-treated mice (p=0.0003 by both log-rank [Mantel-Cox] and Gehan-Breslow-Wilcoxon test). N=14-15/group.

FIGS. 18A-B. Lower motor neurons in the lumbar spine were identified by ChAT staining (FIG. 18A) and quantified by level by two independent, treatment-blinded reviewers (FIG. 18B). TAR+/4 mice treated with miATXN2-V1 or buffer were compared to wild-type littermates. Lower motor neuron counts were significantly increased in the distal (L6) region of the lumbar spine. N=2-3/group.

FIGS. 19A-D. Inflammation in the frontal cortex was measured at three weeks of age by staining for astrocytes (GFPA, FIGS. 19A-B) and microglia (Iba1, FIGS. 19C-D). miATXN2-treated TAR^{4/4} mice showed a trend towards astrogliosis (FIG. 19B) and significantly decreased microgliosis (FIG. 19D) compared to buffer-treated TAR^{4/4} mice. N=3/group.

FIGS. 20A-B. Phosphorylated TDP-43, a measure of ALS pathology with high variability in mutant mice, was measured in the lumbar spinal cord at three weeks of age (FIG. 20A). miATXN2-treated TAR^{4/4} mice showed decreased phosphorylated TDP-43 (p<0.005) compared to buffer-treated TAR^{4/4} mice (FIG. 20B). N=3-5/group.

FIGS. 21A-E. The inventors analyzed transcriptional changes in their TAR^{4/4} mice compared to wild-type and miATXN2-treated TAR^{4/4} littermates. Mice were treated with buffer or miATNX2-V1 at postnatal day 1 and lumbar spinal cords were harvested at postnatal day 19 and RNA was extracted and measured by bulk sequencing. Mutant mice showed dramatic global transcriptional changes compared to wildtype, with 2302 genes showing at least a 0.5-fold change at cutoff adjusted p-value of <0.05 (FIG. 21A). 153 of those genes corrected when miATXN2-treated mice were compared to wildtype (FIG. 21B, blue dots), defined as differing by <0.05-fold and having an adjusted p-value>0.05. They also looked at the total genes that were significantly altered in TAR^{4/4} mice by miATXN2 treatment (FIG. 21C), of which 32 genes were corrected to the wildtype levels (FIG. 21D). Examples of 9 of those individual genes are shown FIG. 25E. N=5-6/group, balanced within litter.

### DETAILED DESCRIPTION

Here, the inventors used an miRNA screen to identify several miRNAs against ATXN2 as the most effective ones to knock down ATXN2 mRNA in mouse and human immortalized cell lines in vitro. They used AAV to deliver these miRNAs to mouse models of both ALS and SCA2 and have achieved strong knockdown in affected areas of the central nervous system including the motor cortex, brainstem and spinal cord (the ALS-relevant areas) and the cerebellum (the SCA2-relevant areas). Finally, they probed for inflammatory markers and showed that there is no evidence of inflammation after treatment. These and other aspects of the disclosure are discussed in greater detail below.

### I. ATAXIN 2 (ATXN2) AND RELATED DISEASE CONDITIONS

Ataxin-2 is a protein that in humans is encoded by the ATXN2 gene. Mutations in ATXN2 cause spinocerebellar ataxia type 2 (SCA2). Ataxin-2 contains two LSm domains, which likely allow it to bind RNA; a PAM2 motif, predicted to associate with the poly(A)-binding protein; and a polyglutamine tract in some species (located near the amino terminal in primates and between the LSm domains in insects). A potential transcript variant, missing an internal coding exon, has been described; however, its full-length nature is not certain. The following sequences are known:

ATXN2 Transcript variant 1: (Protein coding) NM_002973.4
(SEQ ID NO: 4)
agagctcgcctccctccgcctcagactgttttggtagcaacggcaacggggggcgcgtttcggcccggctc
ccggcggctccttggtctcggcgggcctccccgccccttcgtcgtcctccttctccccctcgccagcccgggcg
cccctccggccgcgccaacccgcgcctccccgctcggcgcccgcgcgtccccgccgcgttccggcgtctcct
tggcgcgcccggctcccggctgtccccgcccggcgtgcgagccggtgtatgggcccctcaccatgtcgctga
agccccagcagcagcagcagcagcagcagcagcagcagcagcagcaacagcagcagcagcagcagcag
cagcagccgccgcccgcggctgccaatgtccgcaagcccggcggcagcggccttctagcgtcgcccgccg
ccgcgccttcgccgtcctcgtcctcggtctcctcgtcctcggccacggctccctcctcggtggtcgcggcgacct
ccggcggcgggaggcccggcctgggcagaggtcgaaacagtaacaaaggactgcctcagtctacgatttcttt
tgatggaatctatgcaaatatgaggatggttcatatacttacatcagttgttggctccaaatgtgaagtacaagtga
aaaatggaggtatatatgaaggagtttttaaaacttacagtccgaagtgtgatttggtacttgatgccgcacatgag
aaaagtacagaatccagttcggggccgaaacgtgaagaaataatggagagtattttgttcaaatgttcagactttg
ttgtggtacagtttaaagatatggactccagttatgcaaaaagagatgcttttactgactctgctatcagtgctaaag
tgaatggcgaacacaaagagaaggacctggagccctgggatgcaggtgaactcacagccaatgaggaacttg
aggctttggaaaatgacgtatctaatggatgggatcccaatgatatgtttcgatataatgaagaaaattatggtgta
gtgtctacgtatgatagcagtttatcttcgtatacagtgcccttagaaagagataactcagaagaatttttaaaacgg
gaagcaagggcaaaccagttagcagaagaaattgagtcaagtgcccagtacaaagctcgagtggccctggaa
aatgatgataggagtgaggaagaaaaatacacagcagttcagagaaattccagtgaacgtgaggggcacagc
ataaacactagggaaaataaatatattcctcctggacaaagaaatagagaagtcatatcctggggaagtgggag
acagaattcaccgcgtatgggccagcctggatcgggctccatgccatcaagatccacttctcacacttcagatttc
aacccgaattctggttcagaccaaagagtagttaatggaggtgttccctggccatcgccttgcccatctccttcctc
tcgcccaccttctcgctaccagtcaggtcccaactctcttccacctcgggcagccacccctacacggccgccct
ccaggcccccctcgcggccatccagacccccgtctcacccctctgctcatggttctccagctcctgtctctactat
gcctaaacgcatgtcttcagaagggcctccaaggatgtccccaaaggcccagcgacatcctcgaaatcacaga
gtttctgctgggaggggttccatatccagtggcctagaatttgtatcccacaacccacccagtgaagcagctactc
ctccagtagcaaggaccagtccctcggggggaacgtggtcatcagtggtcagtggggttccaagattatcccct
aaaactcatagacccaggtctcccagacagaacagtattggaaatacccccagtgggccagttcttgcttctccc
caagctggtattattccaactgaagctgttgccatgcctattccagctgcatctcctacgcctgctagtcctgcatc
gaacagagctgttaccccttctagtgaggctaaagattccaggcttcaagatcagaggcagaactctcctgcag
ggaataaagaaaatattaaacccaatgaaacatcacctagcttctcaaaagctgaaaacaaaggtatatcaccag
ttgtttctgaacatagaaaacagattgatgatttaaagaaatttaagaatgattttaggttacagccaagttctacttct
gaatctatggatcaactactaaacaaaaatagagagggagaaaaatcaagagatttgatcaaagacaaaattga
accaagtgctaaggattctttcattgaaaatagcagcagcaactgtaccagtggcagcagcaagccgaatagcc
ccagcatttccccttcaatacttagtaacacggagcacaagaggggacctgaggtcacttcccaaggggttcag
acttccagcccagcatgtaaacaagagaaagacgataaggaagagaagaaagacgcagctgagcaagttag
gaaatcaacattgaatcccaatgcaaaggagttcaacccacgttccttctctcagccaaagccttctactacccca
acttcacctcggcctcaagcacaacctagcccatctatggtgggtcatcaacagccaactccagtttatactcagc
ctgtttgttttgcaccaaatatgatgtatccagtcccagtgagcccaggcgtgcaacctttatacccaatacctatga
cgcccatgccagtgaatcaagccaagacatatagagcagtaccaaatatgccccaacagcggcaagaccagc
atcatcagagtgccatgatgcacccagcgtcagcagcgggcccaccgattgcagccaccccaccagcttactc
cacgcaatatgttgcctacagtcctcagcagttcccaaatcagccccttgttcagcatgtgccacattatcagtctc
agcatcctcatgtctatagtcctgtaatacagggtaatgctagaatgatggcaccaccaacacacgcccagcctg
gtttagtatcttcttcagcaactcagtacggggctcatgagcagacgcatgcgatgtatgcatgtcccaaattacc
atacaacaaggagacaagcccttctttctactttgccatttccacgggctcccttgctcagcagtatgcgcacccta
acgctaccctgcacccacatactccacaccctcagccttcagctacccccactggacagcagcaaagccaaca
tggtggaagtcatcctgcacccagtcctgttcagcaccatcagcaccaggccgcccaggctctccatctggcca
gtccacagcagcagtcagccatttaccacgcggggcttgcgccaactccaccctccatgacacctgcctccaa
cacgcagtcgccacagaatagtttcccagcagcacaacagactgtctttacgatccatccttctcacgttcagcc
ggcgtataccaacccaccccacatggcccacgtacctcaggctcatgtacagtcaggaatggttccttctcatcc
aactgcccatgcgccaatgatgctaatgacgacacagccacccggcggtccccaggccgccctcgctcaaag
tgcactacagcccattccagtctcgacaacagcgcatttcccctatatgacgcacccttcagtacaagcccacca
ccaacagcagttgtaaggctgccctggaggaaccgaaaggccaaattccctcctcccttctactgcttctaccaa
ctggaagcacagaaaactagaatttcatttattttgtttttaaaatatatatgttgatttcttgtaacatccaataggaat
gctaacagttcacttgcagtggaagatacttggaccgagtagaggcatttaggaacttgggggctattccataatt
ccatatgctgtttcagagtcccgcaggtaccccagctctgcttgccgaaactggaagttatttattttttaataaccct
tgaaagtcatgaacacatcagctagcaaaagaagtaacaagagtgattcttgctgctattactgctaaaaaaaaa
aaaaaaaaaaaatcaagacttggaacgcccttttactaaacttgacaaagtttcagtaaattcttaccgtcaaactg
acggattattatttataaatcaagtttgatgaggtgatcactgtctacagtggttcaacttttaagttaagggaaaaac
ttttactttgtagataatataaaataaaaacttaaaaaaaatttaaaaaaaaaaaaagttttaaaaactga
ATXN2 Transcript variant 2: (Protein coding) NM_001310121 (SEQ ID NO: 5)
cccgagaaagcaacccagcgcgccgcccgctcctcacgtgtccctcccggccccggggccacctcacgttct
gcttccgtctgacccctccgacttccgaggtcgaaacagtaacaaaggactgcctcagtctacgatttcttttgatg
gaatctatgcaaatatgaggatggttcatatacttacatcagttgttggctccaaatgtgaagtacaagtgaaaaat
ggaggtatatatgaaggagtttttaaaacttacagtccgaagtgtgatttggtacttgatgccgcacatgagaaaa
gtacagaatccagttcggggccgaaacgtgaagaaataatggagagtattttgttcaaatgttcagactttgttgtg
gtacagtttaaagatatggactccagttatgcaaaaagagatgcttttactgactctgctatcagtgctaaagtgaat
ggcgaacacaaagagaaggacctggagccctgggatgcaggtgaactcacagccaatgaggaacttgaggc
tttggaaaatgacgtatctaatggatgggatcccaatgatatgtttcgatataatgaagaaaattatggtgtagtgtc
tacgtatgatagcagtttatcttcgtatacagtgcccttagaaagagataactcagaagaatttttaaaacgggaag
caagggcaaaccagttagcagaagaaattgagtcaagtgcccagtacaaagctcgagtggccctggaaaatg
atgataggagtgaggaagaaaaatacacagcagttcagagaaattccagtgaacgtgaggggcacagcataa
acactagggaaaataaatatattcctcctggacaaagaaatagagaagtcatatcctggggaagtgggagacag
aattcaccgcgtatgggccagcctggatcgggctccatgccatcaagatccacttctcacacttcagatttcaacc
cgaattctggttcagaccaaagagtagttaatggaggtgttccctggccatcgccttgcccatctccttcctctcgc
ccaccttctcgctaccagtcaggtcccaactctcttccacctcgggcagccacccctacacggccgccctccag
gcccccctcgcggccatccagacccccgtctcacccctctgctcatggttctccagctcctgtctctactatgcct
aaacgcatgtcttcagaagggcctccaaggatgtccccaaaggcccagcgacatcctcgaaatcacagagtttc
tgctgggaggggttccatatccagtggcctagaatttgtatcccacaacccacccagtgaagcagctactcctcc
agtagcaaggaccagtccctcggggggaacgtggtcatcagtggtcagtggggttccaagattatcccctaaa
actcatagacccaggtctcccagacagaacagtattggaaatacccccagtgggccagttcttgcttctccccaa
gctggtattattccaactgaagctgttgccatgcctattccagctgcatctcctacgcctgctagtcctgcatcgaa
cagagctgttaccccttctagtgaggctaaagattccaggcttcaagatcagaggcagaactctcctgcaggga
ataaagaaaatattaaacccaatgaaacatcacctagcttctcaaaagctgaaaacaaaggtatatcaccagttgt
ttctgaacatagaaaacagattgatgatttaaagaaatttaagaatgattttaggttacagccaagttctacttctgaa
tctatggatcaactactaaacaaaaatagagagggagaaaaatcaagagatttgatcaaagacaaaattgaacc
aagtgctaaggattctttcattgaaaatagcagcagcaactgtaccagtggcagcagcaagccgaatagcccca
gcatttccccttcaatacttagtaacacggagcacaagaggggacctgaggtcacttcccaaggggttcagactt
ccagcccagcatgtaaacaagagaaagacgataaggaagagaagaaagacgcagctgagcaagttaggaa
atcaacattgaatcccaatgcaaaggagttcaacccacgttccttctctcagccaaagccttctactaccccaactt
cacctcggcctcaagcacaacctagcccatctatggtgggtcatcaacagccaactccagtttatactcagcctg
tttgttttgcaccaaatatgatgtatccagtcccagtgagcccaggcgtgcaacctttatacccaatacctatgacg
cccatgccagtgaatcaagccaagacatatagagcagtaccaaatatgccccaacagcggcaagaccagcat
catcagagtgccatgatgcacccagcgtcagcagcgggcccaccgattgcagccaccccaccagcttactcc
acgcaatatgttgcctacagtcctcagcagttcccaaatcagccccttgttcagcatgtgccacattatcagtctca
gcatcctcatgtctatagtcctgtaatacagggtaatgctagaatgatggcaccaccaacacacgcccagcctgg
tttagtatcttcttcagcaactcagtacggggctcatgagcagacgcatgcgatgtatgcatgtcccaaattaccat
acaacaaggagacaagcccttctttctactttgccatttccacgggctcccttgctcagcagtatgcgcaccctaa
cgctaccctgcacccacatactccacaccctcagccttcagctacccccactggacagcagcaaagccaacat
ggtggaagtcatcctgcacccagtcctgttcagcaccatcagcaccaggccgcccaggctctccatctggcca
gtccacagcagcagtcagccatttaccacgcggggcttgcgccaactccaccctccatgacacctgcctccaa
cacgcagtcgccacagaatagtttcccagcagcacaacagactgtctttacgatccatccttctcacgttcagcc
ggcgtataccaacccaccccacatggcccacgtacctcagtgcgccagtgaggctctggcaaggtgtgggcta
gagatgcgactcagttggatctatctctcagaaggctaccttgctcatgtacagtcaggaatggttccttctcatcc
aactgcccatgcgccaatgatgctaatgacgacacagccacccggcggtccccaggccgccctcgctcaaag
tgcactacagcccattccagtctcgacaacagcgcatttcccctatatgacgcacccttcagtacaagcccacca
ccaacagcagttgtaaggctgccctggaggaaccgaaaggccaaattccctcctcccttctactgcttctaccaa
ctggaagcacagaaaactagaatttcatttattttgtttttaaaatatatatgttgatttcttgtaacatccaataggaat
gctaacagttcacttgcagtggaagatacttggaccgagtagaggcatttaggaacttgggggctattccataatt
ccatatgctgtttcagagtcccgcaggtaccccagctctgcttgccgaaactggaagttatttattttttaataaccct
tgaaagtcatgaacacatcagctagcaaaagaagtaacaagagtgattcttgctgctattactgctaaaaaaaaa
aaaaaaaaaaaatcaagacttggaacgcccttttactaaacttgacaaagtttcagtaaattcttaccgtcaaactg
acggattattatttataaatcaagtttgatgaggtgatcactgtctacagtggttcaacttttaagttaagggaaaaac
ttttactttgtagataatataaaataaaaacttaaaaaaaatttaaaaaataaaaaaagttttaaaaactgaaaaaaaa
aaa
ATXN2 Transcript variant 3: (Protein coding) NM_001310123
(SEQ ID NO: 6)
cccgagaaagcaacccagcgcgccgcccgctcctcacgtgtccctcccggccccggggccacctcacgttct
gcttccgtctgacccctccgacttccgatttcttttgatggaatctatgcaaatatgaggatggttcatatacttacat
cagttgtttgtgatttggtacttgatgccgcacatgagaaaagtacagaatccagttcggggccgaaacgtgaag
aaataatggagagtattttgttcaaatgttcagactttgttgtggtacagtttaaagatatggactccagttatgcaaa
aagagatgcttttactgactctgctatcagtgctaaagtgaatggcgaacacaaagagaaggacctggagccct
gggatgcaggtgaactcacagccaatgaggaacttgaggctttggaaaatgacgtatctaatggatgggatccc
aatgatatgtttcgatataatgaagaaaattatggtgtagtgtctacgtatgatagcagtttatcttcgtatacagtgc
ccttagaaagagataactcagaagaatttttaaaacgggaagcaagggcaaaccagttagcagaagaaattga
gtcaagtgcccagtacaaagctcgagtggccctggaaaatgatgataggagtgaggaagaaaaatacacagc
agttcagagaaattccagtgaacgtgaggggcacagcataaacactagggaaaataaatatattcctcctggac
aaagaaatagagaagtcatatcctggggaagtgggagacagaattcaccgcgtatgggccagcctggatcgg
gctccatgccatcaagatccacttctcacacttcagatttcaacccgaattctggttcagaccaaagagtagttaat
ggaggtgttccctggccatcgccttgcccatctccttcctctcgcccaccttctcgctaccagtcaggtcccaact
ctcttccacctcgggcagccacccctacacggccgccctccaggcccccctcgcggccatccagacccccgt
ctcacccctctgctcatggttctccagctcctgtctctactatgcctaaacgcatgtcttcagaagggcctccaagg
atgtccccaaaggcccagcgacatcctcgaaatcacagagtttctgctgggaggggttccatatccagtggcct
agaatttgtatcccacaacccacccagtgaagcagctactcctccagtagcaaggaccagtccctcgggggga
acgtggtcatcagtggtcagtggggttccaagattatcccctaaaactcatagacccaggtctcccagacagaac
agtattggaaatacccccagtgggccagttcttgcttctccccaagctggtattattccaactgaagctgttgccat
gcctattccagctgcatctcctacgcctgctagtcctgcatcgaacagagctgttaccccttctagtgaggctaaa
gattccaggcttcaagatcagaggcagaactctcctgcagggaataaagaaaatattaaacccaatgaaacatc
acctagcttctcaaaagctgaaaacaaaggtatatcaccagttgtttctgaacatagaaaacagattgatgatttaa
agaaatttaagaatgattttaggttacagccaagttctacttctgaatctatggatcaactactaaacaaaaatagag
agggagaaaaatcaagagatttgatcaaagacaaaattgaaccaagtgctaaggattctttcattgaaaatagca
gcagcaactgtaccagtggcagcagcaagccgaatagccccagcatttccccttcaatacttagtaacacggag
cacaagaggggacctgaggtcacticccaaggggttcagacliccagcccagcatgtaaacaagagaaagac
gataaggaagagaagaaagacgcagctgagcaagttaggaaatcaacattgaatcccaatgcaaaggagttc
aacccacgttccttctctcagccaaagccttctactaccccaacttcacctcggcctcaagcacaacctagcccat
ctatggtgggtcatcaacagccaactccagtttatactcagcctgtttgttttgcaccaaatatgatgtatccagtcc
cagtgagcccaggcgtgcaacctttatacccaatacctatgacgcccatgccagtgaatcaagccaagacatat
agagcagtaccaaatatgccccaacagcggcaagaccagcatcatcagagtgccatgatgcacccagcgtca
gcagcgggcccaccgattgcagccaccccaccagcttactccacgcaatatgttgcctacagtcctcagcagtt
cccaaatcagccccttgttcagcatgtgccacattatcagtctcagcatcctcatgtctatagtcctgtaatacagg
gtaatgctagaatgatggcaccaccaacacacgcccagcctggtttagtatcttcttcagcaactcagtacgggg
ctcatgagcagacgcatgcgatgtatgtttccacgggctcccttgctcagcagtatgcgcaccctaacgctaccc
tgcacccacatactccacaccctcagccttcagctacccccactggacagcagcaaagccaacatggtggaag
tcatcctgcacccagtcctgttcagcaccatcagcaccaggccgcccaggctctccatctggccagtccacagc
agcagtcagccatttaccacgcggggcttgcgccaactccaccctccatgacacctgcctccaacacgcagtc
gccacagaatagtttcccagcagcacaacagactgtctttacgatccatccttctcacgttcagccggcgtatacc
aacccaccccacatggcccacgtacctcaggctcatgtacagtcaggaatggttccttctcatccaactgcccat
gcgccaatgatgctaatgacgacacagccacccggcggtccccaggccgccctcgctcaaagtgcactacag
cccattccagtctcgacaacagcgcatttcccctatatgacgcacccttcagtacaagcccaccaccaacagca
gttgtaaggctgccctggaggaaccgaaaggccaaattccctcctcccttctactgcttctaccaactggaagca
cagaaaactagaatttcatttattttgtttttaaaatatatatgttgatttcttgtaacatccaataggaatgctaacagtt
cacttgcagtggaagatacttggaccgagtagaggcatttaggaacttgggggctattccataattccatatgctg
tttcagagtcccgcaggtaccccagctctgcttgccgaaactggaagttatttattttttaataacccttgaaagtcat
gaacacatcagctagcaaaagaagtaacaagagtgattcttgctgctattactgctaaaaaaaaaaaaaaaaaaa
aatcaagacttggaacgcccttttactaaacttgacaaagtttcagtaaattcttaccgtcaaactgacggattattat
ttataaatcaagtttgatgaggtgatcactgtctacagtggttcaacttttaagttaagggaaaaacttttactttgtag
ataatataaaataaaaacttaaaaaaaatttaaaaaaaaaaaaagttttaaaaactgaaaaaaaaaaa
ATXN2 Transcript variant 4 (Non-Coding RNA): NR_132311
(SEQ ID NO: 7)
agagctcgcctccctccgcctcagactgttttggtagcaacggcaacggcggcggcgcgtttcggcccggctc
ccggggctccttggtctcgggggcctccccgccccttcgtcgtcctccttctccccctcgccagcccgggcg
cccctccggccgcgccaacccgcgcctccccgctcggcgcccgcgcgtccccgccgcgttccggcgtctcct
tggcgcgcccggctcccggctgtccccgcccggcgtgcgagccggtgtatgggcccctcaccatgtcgctga
agccccagcagcagcagcagcagcagcagcagcagcagcagcagcaacagcagcagcagcagcagcag
cagcagccgccgcccgcggctgccaatgtccgcaagcccggcggcagcggccttctagcgtcgcccgccg
ccgcgccttcgccgtcctcgtcctcggtctcctcgtcctcggccacggctccctcctcggtggtcgcggcgacct
ccggcggcgggaggcccggcctgggcagaggtcgaaacagtaacaaaggactgcctcagtctacgatttcttt
tgatggaatctatgcaaatatgaggatggttcatatacttacatcagttgttggctccaaatgtgaagtacaagtga
aaaatggaggtatatatgaaggagtttttaaaacttacagtccgaagtgtgatttggtacttgatgccgcacatgag
aaaagtacagaatccagttcggggccgaaacgtgaagaaataatggagagtattttgttcaaatgttcagactttg
ttgtggtacagtttaaagatatggactccagttatgcaaaaagagatgcttttactgactctgctatcagtgctaaag
tgaatggcgaacacaaagagaaggacctggagccctgggatgcaggtgaactcacagccaatgaggaacttg
aggctttggaaaatgacgtatctaatggatgggatcccaatgatatgtttcgatataatgaagaaaattatggtgta
gtgtctacgtatgatagcagtttatcttcgtatacagtgcccttagaaagagataactcagaagaatttttaaaacgg
gaagcaagggcaaaccagttagcagaagaaattgagtcaagtgcccagtacaaagctcgagtggccctggaa
aatgatgataggagtgaggaagaaaaatacacagcagttcagagaaattccagtgaacgtgaggggcacagc
ataaacactagggaaaataaatatattcctcctggacaaagaaatagagaagtcatatcctggggaagtgggag
acagaattcaccgcgtatgggccagcctggatcgggctccatgccatcaagatccacttctcacacttcagatttc
aacccgaattctggttcagaccaaagagtagttaatggaggtgttccctggccatcgccttgcccatctccttcctc
tcgcccaccttctcgctaccagtcaggtcccaactctcttccacctcgggcagccacccctacacggccgccct
ccaggcccccctcgcggccatccagacccccgtctcacccctctgctcatggttctccagctcctgtctctactat
gcctaaacgcatgtcttcagaagggcctccaaggatgtccccaaaggcccagcgacatcctcgaaatcacaga
gtttctgctgggaggggttccatatccagtggcctagaatttgtatcccacaacccacccagtgaagcagctactc
ctccagtagcaaggaccagtccctcggggggaacgtggtcatcagtggtcagtggggttccaagattatcccct
aaaactcatagacccaggtctcccagacagaacagtattggaaatacccccagtgggccagttcttgcttctccc
caagctggtattattccaactgaagctgttgccatgcctattccagctgcatctcctacgcctgctagtcctgcatc
gaacagagctgttaccccttctagtgaggctaaagattccaggcttcaagatcagaggcagaactctcctgcag
ggaataaagaaaatattaaacccaatgaaacatcacctagcttctcaaaagctgaaaacaaaggtatatcaccag
ttgtttctgaacatagaaaacagattgatgatttaaagaaatttaagaatgattttaggttacagccaagttctacttct
gaatctatggatcaactactaaacaaaaatagagagggagaaaaatcaagagatttgatcaaagacaaaattga
accaagtgctaaggattctttcattgaaaatagcagcagcaactgtaccagtggcagcagcaagccgaatagcc
ccagcatttccccttcaatacttagtaacacggagcacaagaggggacctgaggtcacttcccaaggggttcag
acttccagcccagcatgtaaacaagagaaagacgataaggaagagaagaaagacgcagctgagcaagttag
gaaatcaacattgaatcccaatgcaaaggagttcaacccacgttccttctctcagccaaagccttctactacccca
acttcacctcggcctcaagcacaacctagcccatctatggtgggtcatcaacagccaactccagtttatactcagc
ctgtttgttttgcaccaaatatgatgtatccagtcccagtgagcccaggcgtgcaataccaaatatgccccaacag
cggcaagaccagcatcatcagagtgccatgatgcacccagcgtcagcagcgggcccaccgattgcagccac
cccaccagcttactccacgcaatatgttgcctacagtcctcagcagttcccaaatcagccccttgttcagcatgtg
ccacattatcagtctcagcatcctcatgtctatagtcctgtaatacagggtaatgctagaatgatggcaccaccaac
acacgcccagcctggtttagtatcttcttcagcaactcagtacggggctcatgagcagacgcatgcgatgtatgc
atgtcccaaattaccatacaacaaggagacaagcccttctttctactttgccatttccacgggctcccttgctcagc
agtatgcgcaccctaacgctaccctgcacccacatactccacaccctcagccttcagctacccccactggacag
cagcaaagccaacatggtggaagtcatcctgcacccagtcctgttcagcaccatcagcaccaggccgcccag
gctctccatctggccagtccacagcagcagtcagccatttaccacgcggggcttgcgccaactccaccctccat
gacacctgcctccaacacgcagtcgccacagaatagtttcccagcagcacaacagactgtctttacgatccatc
cttctcacgttcagccggcgtataccaacccaccccacatggcccacgtacctcaggctcatgtacagtcagga
atggttccttctcatccaactgcccatgcgccaatgatgctaatgacgacacagccacccggggtccccaggc
cgccctcgctcaaagtgcactacagcccattccagtctcgacaacagcgcatttcccctatatgacgcacccttc
agtacaagcccaccaccaacagcagttgtaaggctgccctggaggaaccgaaaggccaaattccctcctccct
tctactgcttctaccaactggaagcacagaaaactagaatttcatttattttgtttttaaaatatatatgttgatttcttgta
acatccaataggaatgctaacagttcacttgcagtggaagatacttggaccgagtagaggcatttaggaacttgg
gggctattccataattccatatgctgtttcagagtcccgcaggtaccccagctctgcttgccgaaactggaagttat
ttattttttaataacccttgaaagtcatgaacacatcagctagcaaaagaagtaacaagagtgattcttgctgctatta
ctgctaaaaaaaaaaaaaaaaaaaaatcaagacttggaacgcccttttactaaacttgacaaagtttcagtaaatt
cttaccgtcaaactgacggattattatttataaatcaagtttgatgaggtgatcactgtctacagtggttcaacttttaa
gttaagggaaaaacttttactttgtagataatataaaataaaaacttaaaaaaaatttaaaaaataaaaaaagttttaa
aaactga
ATXN2 Transcript variant 5: (Protein coding) NM_001372574
(SEQ ID NO: 8)
agagctcgcctccctccgcctcagactgttttggtagcaacggcaacggcggcggcgcgtttcggcccggctc
ccggcggctccttggtctcgggggcctccccgccccttcgtcgtcctccttctccccctcgccagcccgggcg
cccctccggccgcgccaacccgcgcctccccgctcggcgcccgcgcgtccccgccgcgttccggcgtctcct
tggcgcgcccggctcccggctgtccccgcccggcgtgcgagccggtgtatgggcccctcaccatgtcgctga
agccccagcagcagcagcagcagcagcagcagcagcagcagcagcaacagcagcagcagcagcagcag
cagcagccgccgcccgcggctgccaatgtccgcaagcccggcggcagcggccttctagcgtcgcccgccg
ccgcgccttcgccgtcctcgtcctcggtctcctcgtcctcggccacggctccctcctcggtggtcgcggcgacct
ccggcggcgggaggcccggcctgggcagaggtcgaaacagtaacaaaggactgcctcagtctacgatttcttt
tgatggaatctatgcaaatatgaggatggttcatatacttacatcagttgttggctccaaatgtgaagtacaagtga
aaaatggaggtatatatgaaggagtttttaaaacttacagtccgaagtgtgatttggtacttgatgccgcacatgag
aaaagtacagaatccagttcggggccgaaacgtgaagaaataatggagagtattttgttcaaatgttcagactttg
ttgtggtacagtttaaagatatggactccagttatgcaaaaagagatgcttttactgactctgctatcagtgctaaag
tgaatggcgaacacaaagagaaggacctggagccctgggatgcaggtgaactcacagccaatgaggaacttg
aggctttggaaaatgacgtatctaatggatgggatcccaatgatatgtttcgatataatgaagaaaattatggtgta
gtgtctacgtatgatagcagtttatcttcgtatacagtgcccttagaaagagataactcagaagaatttttaaaacgg
gaagcaagggcaaaccagttagcagaagaaattgagtcaagtgcccagtacaaagctcgagtggccctggaa
aatgatgataggagtgaggaagaaaaatacacagcagttcagagaaattccagtgaacgtgaggggcacagc
ataaacactagggaaaataaatatattcctcctggacaaagaaatagagaagtcatatcctggggaagtgggag
acagaattcaccgcgtatgggccagcctggatcgggctccatgccatcaagatccacttctcacacttcagatttc
aacccgaattctggttcagaccaaagagtagttaatggaggtgttccctggccatcgccttgcccatctccttcctc
tcgcccaccttctcgctaccagtcaggtcccaactctcttccacctcgggcagccacccctacacggccgccct
ccaggcccccctcgcggccatccagacccccgtctcacccctctgctcatggttctccagctcctgtctctactat
gcctaaacgcatgtcttcagaagggcctccaaggatgtccccaaaggcccagcgacatcctcgaaatcacaga
gtttctgctgggaggggttccatatccagtggcctagaatttgtatcccacaacccacccagtgaagcagctactc
ctccagtagcaaggaccagtccctcggggggaacgtggtcatcagtggtcagtggggttccaagattatcccct
aaaactcatagacccaggtctcccagacagaacagtattggaaatacccccagtgggccagttcttgcttctccc
caagctggtattattccaactgaagctgttgccatgcctattccagctgcatctcctacgcctgctagtcctgcatc
gaacagagctgttaccccttctagtgaggctaaagattccaggcttcaagatcagaggcagaactctcctgcag
ggaataaagaaaatattaaacccaatgaaacatcacctagcttctcaaaagctgaaaacaaaggtatatcaccag
ttgtttctgaacatagaaaacagattgatgatttaaagaaatttaagaatgattttaggttacagccaagttctacttct
gaatctatggatcaactactaaacaaaaatagagagggagaaaaatcaagagatttgatcaaagacaaaattga
accaagtgctaaggattctttcattgaaaatagcagcagcaactgtaccagtggcagcagcaagccgaatagcc
ccagcatttccccttcaatacttagtaacacggagcacaagaggggacctgaggtcacttcccaaggggttcag
acttccagcccagcatgtaaacaagagaaagacgataaggaagagaagaaagacgcagctgagcaagttag
gaaatcaacattgaatcccaatgcaaaggagttcaacccacgttccttctctcagccaaagccttctactacccca
acttcacctcggcctcaagcacaacctagcccatctatggtgggtcatcaacagccaactccagtttatactcagc
ctgtttgttttgcaccaaatatgatgtatccagtcccagtgagcccaggcgtgcaacctttatacccaatacctatga
cgcccatgccagtgaatcaagccaagacatatagagcaggtaaagtaccaaatatgccccaacagcggcaag
accagcatcatcagagtgccatgatgcacccagcgtcagcagcgggcccaccgattgcagccaccccaccag
cttactccacgcaatatgttgcctacagtcctcagcagttcccaaatcagccccttgttcagcatgtgccacattatc
agtctcagcatcctcatgtctatagtcctgtaatacagggtaatgctagaatgatggcaccaccaacacacgccc
agcctggtttagtatcttcttcagcaactcagtacggggctcatgagcagacgcatgcgatgtatgcatgtcccaa
attaccatacaacaaggagacaagcccttctttctactttgccatttccacgggctcccttgctcagcagtatgcgc
accctaacgctaccctgcacccacatactccacaccctcagccttcagctacccccactggacagcagcaaag
ccaacatggtggaagtcatcctgcacccagtcctgttcagcaccatcagcaccaggccgcccaggctctccatc
tggccagtccacagcagcagtcagccatttaccacgcggggcttgcgccaactccaccctccatgacacctgc
ctccaacacgcagtcgccacagaatagtttcccagcagcacaacagactgtctttacgatccatccttctcacgtt
cagccggcgtataccaacccaccccacatggcccacgtacctcaggctcatgtacagtcaggaatggttccttc
tcatccaactgcccatgcgccaatgatgctaatgacgacacagccacccggcggtccccaggccgccctcgct
caaagtgcactacagcccattccagtctcgacaacagcgcatttcccctatatgacgcacccttcagtacaagcc
caccaccaacagcagttgtaaggctgccctggaggaaccgaaaggccaaattccctcctcccttctactgcttct
accaactggaagcacagaaaactagaatttcatttattttgtttttaaaatatatatgttgatttcttgtaacatccaata
ggaatgctaacagttcacttgcagtggaagatacttggaccgagtagaggcatttaggaacttgggggctattcc
ataattccatatgctgtttcagagtcccgcaggtaccccagctctgcttgccgaaactggaagttatttattttttaat
aacccttgaaagtcatgaacacatcagctagcaaaagaagtaacaagagtgattcttgctgctattactgctaaaa
aaaaaaaaaaaaaaaaatcaagacttggaacgcccttttactaaacttgacaaagtttcagtaaattcttaccgtca
aactgacggattattatttataaatcaagtttgatgaggtgatcactgtctacagtggttcaacttttaagttaaggga
aaaacttttactttgtagataatataaaataaaaacttaaaaaaaatttaaaaaataaaaaaagttttaaaaactga

ATXN2 is conserved across eukaryotes. Most vertebrates have two orthologs of the gene (called ATXN2 and ATXN2L in humans), with the exception of birds which only have one. Plant species have two to six ATXN2 orthologs. ATXN2 is ubiquitously expressed in different tissues. Within individual cells, it localizes to the Golgi apparatus and stress granules. Ataxin-2 is involved in regulating mRNA translation through its interactions with the poly(A)-binding protein. It is also involved in the formation of stress granules and P-bodies, which also play roles in RNA regulation.

The polyglutamine tract in human ataxin-2 is unstable and can expand as it is transmitted across generations. Normal alleles usually have 22 or 23 repeats but can contain up to 31 repeats. Longer expansions can cause spinocerebellar ataxia type 2 (SCA2), a fatal progressive genetic disorder in which neurons degenerate in the cerebellum, inferior olive, pons, and other areas. Symptoms of SCA2 include ataxia (a loss of coordinated movements), parkinsonism, and dementia in some cases. The disease allele usually contains 34-52 CAG repeats, but can contain as few as 32 or more than 100, and can expand in size when transmitted to successive generations. How the polyglutamine expansion in ataxin-2 leads to these symptoms is unknown. It has also been shown that intermediate-size CAG repeat expansions are significantly associated with risk for developing amyotrophic lateral sclerosis (Lou Gehrig's disease).

### A. Spinocerebellar Ataxia Type 2

Spinocerebellar ataxia (SCA) is one of a group of genetic disorders characterized by slowly progressive incoordination of gait and is often associated with poor coordination of hands, speech, and eye movements. A review of different clinical features among SCA subtypes was recently published describing the frequency of non-cerebellar features, like parkinsonism, chorea, cognitive impairment, peripheral neuropathy, and seizures, among others. As with other forms of ataxia, SCA results in atrophy of the cerebellum, loss of fine coordination of muscle movements leading to unsteady and clumsy motion, and other symptoms. The symptoms of ataxias vary with the specific type and with the individual patient. In many cases a person with ataxia retains full mental capacity but progressively loses physical control.

The hereditary ataxias are categorized by mode of inheritance and causative gene or chromosomal locus. The hereditary ataxias can be inherited in an autosomal dominant, autosomal recessive, or X-linked manner. Many types of autosomal dominant cerebellar ataxias for which specific genetic information is available are now known. Synonyms for autosomal-dominant cerebellar ataxias (ADCA) used prior to the current understanding of the molecular genetics were Marie's ataxia, inherited olivopontocerebellar atrophy, cerebello-olivary atrophy, or the more generic term “spinocerebellar degeneration.” (Spinocerebellar degeneration is a rare inherited neurological disorder of the central nervous system characterized by the slow degeneration of certain areas of the brain. There are three forms of spinocerebellar degeneration: Types 1, 2, 3. Symptoms begin during adulthood.)

Spinocerebellar ataxia 2 (SCA2) is a progressive disorder that causes symptoms including uncoordinated movement (ataxia), speech and swallowing difficulties, muscle wasting, slow eye movement, and sometimes dementia. Signs and symptoms usually begin in mid-adulthood but can appear any time from childhood to late adulthood. SCA2 is caused by mutations in the ATXN2 gene and is inherited in an autosomal dominant manner.

Early symptoms of spinocerebellar ataxia may include uncoordinated movement (ataxia) and leg cramps. Other symptoms may include tremor; decreased muscle tone; poor tendon reflexes; abnormal eye movements; dementia; dystonia and/or chorea; muscle twitches; nerve irritation and swelling (polyneuropathy); leg weakness; difficulty swallowing; bladder dysfunction; and parkinsonism.

Spinocerebellar ataxia 2 (SCA2) is inherited in an autosomal dominant manner. This means that having one changed (mutated) copy of ATXN2 in each cell is enough to cause signs and symptoms of the condition. The ATXN2 gene mutations that cause SCA2 involve a DNA sequence called a ‘CAG trinucleotide repeat.’ It is made up of a series of three DNA building blocks (CAG stands for cytosine, adenine, and guanine) that appear multiple times in a row. The CAG sequence is normally repeated about 22 times in the gene, but it can be repeated up to 26 or 27 times. Repeats ranging from 26 or 27 to 32 are associated with the development of ALS. SCA2 develops in people who have 33 or more CAG repeats in the ATXN2 gene.

In most cases, an affected person inherits the mutated gene (with too many repeats) from an affected parent. However, in some cases, an affected person does not have an affected parent. People with an increased number of CAG repeats who don't develop SCA2 are still at risk of having children who will develop the disorder. This is because as the gene is passed down from parent to child, the number of CAG repeats often increases. In general, the more repeats a person has, the earlier symptoms begin. This phenomenon is called anticipation. People with 32 or 33 repeats tend to develop symptoms in late adulthood, while people with more than 45 repeats often have symptoms by their teens. For some reason, the number of repeats tends to increase more when the gene is inherited from a person's father than when inherited from a person's mother. Each child of an affected person has a 50% chance of inheriting the CAG repeat expansion.

Molecular genetic testing (analysis of DNA) is needed for a diagnosis of spinocerebellar ataxia 2 (SCA2). This testing detects abnormal CAG trinucleotide repeat expansions in the ATXN2 gene. Affected people (or people who will later develop symptoms of SCA2) have a copy of the ATXN2 gene that has 33 or more CAG repeats. This testing detects nearly 100% of cases of SCA2.

There is no cure for spinocerebellar ataxias, which are currently considered to be progressive and irreversible diseases, although not all types cause equally severe disability. In general, treatments are directed towards alleviating symptoms, not the disease itself. Many patients with hereditary or idiopathic forms of ataxia have other symptoms in addition to ataxia. Medications or other therapies might be appropriate for some of these symptoms, which could include tremor, stiffness, depression, spasticity, and sleep disorders, among others. Both onset of initial symptoms and duration of disease are variable. If the disease is caused by a polyglutamine trinucleotide repeat CAG expansion, a longer expansion may lead to an earlier onset and a more radical progression of clinical symptoms. Typically, a person afflicted with this disease will eventually be unable to perform daily tasks (ADLs). However, rehabilitation therapists can help patients to maximize their ability of self-care and delay deterioration to certain extent. Researchers are exploring multiple avenues for a cure including RNAi and the use of Stem Cells and several other avenues.

Physical therapists can assist patients in maintaining their level of independence through therapeutic exercise programs. One recent research report demonstrated a gain of 2 SARA points (Scale for the Assessment and Rating of Ataxia) from physical therapy. In general, physical therapy emphasizes postural balance and gait training for ataxia patients. General conditioning such as range-of-motion exercises and muscle strengthening would also be included in therapeutic exercise programs. Research showed that spinocerebellar ataxia 2 (SCA2) patients with a mild stage of the disease gained significant improvement in static balance and neurological indices after six months of a physical therapy exercise training program. Occupational therapists may assist patients with incoordination or ataxia issues through the use of adaptive devices. Such devices may include a cane, crutches, walker, or wheelchair for those with impaired gait. Other devices are available to assist with writing, feeding, and self-care if hand and arm coordination are impaired. A randomized clinical trial revealed that an intensive rehabilitation program with physical and occupational therapies for patients with degenerative cerebellar diseases can significantly improve functional gains in ataxia, gait, and activities of daily living. Some level of improvement was shown to be maintained 24 weeks post-treatment. Speech language pathologists may use both behavioral intervention strategies as well as augmentative and alternative communication devices to help patients with impaired speech.

In particular, treatment of spinocerebellar ataxia 2 (SCA2) is supportive and aims to help the affected person maintain their independence and avoid injury. It is recommended that people with SCA2 remain physically active, maintain a healthy weight, use adaptive equipment as needed, and avoid alcohol and medications that affect cerebellar function. Adaptive equipment may include canes or other devices to help with walking and mobility. People with SCA2 may develop difficulty speaking and may need to use computerized devices or writing pads to help with communication. Levodopa may be prescribed to help with some of the movement problems (e.g., rigidity and tremor), and magnesium may improve muscle cramping.

### B. Amyotrophic Lateral Sclerosis

Amyotrophic lateral sclerosis (ALS), sometimes called Lou Gehrig's Disease, affects as many as 20,000 Americans at any given time, with 5,000 new cases being diagnosed in the United States each year. ALS affects people of all races and ethnic backgrounds. Men are about 1.5 times more likely than women to be diagnosed with the disease. ALS strikes in the prime of life, with people most commonly diagnosed between the ages of 40 and 70. However, it is possible for individuals to be diagnosed at younger and older ages. About 90% of ALS cases occur at random, meaning that individuals do not have a family history of the disease. In about 10% of ALS cases there is a family history of the disease.

ALS is a progressive neurological disease that attacks neurons that control voluntary muscles. Motor neurons, which are lost in ALS, are specialized nerve cells located in the brain, brainstem, and spinal cord. These neurons serve as connections from the nervous system to the muscles in the body, and their function is necessary for normal muscle movement. ALS causes motor neurons in both the brain and spinal cord to degenerate, and thus lose the ability to initiate and send messages to the muscles in the body. When the muscles become unable to function, they gradually atrophy and twitch. ALS can begin with very subtle symptoms such as weakness in affected muscles. Where this weakness first appears differs for different people, but the weakness and atrophy spread to other parts of the body as the disease progresses.

Initial symptoms may affect only one leg or arm, causing awkwardness and stumbling when walking or running. Subjects also may suffer difficulty lifting objects or with tasks that require manual dexterity. Eventually, the individual will not be able to stand or walk or use hands and arms to perform activities of daily living. When the muscles in the diaphragm and chest wall become too weak, patients require a ventilator to breathe, although bulbar symptoms can occur at any time, including very early on. Most people with ALS die from respiratory failure, usually 3 to 5 years after being diagnosed; however, some people survive 10 or more years after diagnosis.

Perhaps the most tragic irony of ALS is that in most cases it does not impair a person's mind, as the disease affects only the motor neurons. Personality, intelligence, memory, and self-awareness are sometimes affected, nor are the senses of sight, smell, touch, hearing, and taste. Yet at the same time, ALS can cause dramatic defects in an individual's ability to speak, eventually rendering him or her dependent on eye-tracking technologies for communication. Patients also experience difficulty chewing and swallowing, which increases over time to the point that a feeding tube is required.

### II. miRNAs

In 2001, several groups used a novel cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets.

miRNAs are transcribed by RNA polymerase II and can be derived from individual miRNA genes, from introns of protein coding genes, or from poly-cistronic transcripts that often encode multiple, closely related miRNAs. Pre-miRNAs, generally several thousand bases long, are processed in the nucleus by the RNase Drosha into 70- to 100-nt hairpin-shaped precursors. Following transport to the cytoplasm, the hairpin is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The 5′ portion of a miRNA spanning bases 2-8, termed the ‘seed’ region, is especially important for target recognition (Krenz and Robbins, 2004; Kiriazis and Krania, 2000). The sequence of the seed, together with phylogenetic conservation of the target sequence, forms the basis for many current target prediction models. Although increasingly sophisticated computational approaches to predict miRNAs and their targets are becoming available, target prediction remains a major challenge and requires experimental validation. Ascribing the functions of miRNAs to the regulation of specific mRNA targets is further complicated by the ability of individual miRNAs to base pair with hundreds of potential high and low affinity mRNA targets and by the targeting of multiple miRNAs to individual mRNAs.

The first miRNAs were identified as regulators of developmental timing in C. elegans, suggesting that miRNAs, in general, might play decisive regulatory roles in transitions between different developmental states by switching off specific targets (Fatkin et al., 2000; Lowes et al., 1997). However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression.

Characterizing the functions of biomolecules like miRNAs often involves introducing the molecules into cells or removing the molecules from cells and measuring the result. If introducing a miRNA into cells results in apoptosis, then the miRNA undoubtedly participates in an apoptotic pathway. Methods for introducing and removing miRNAs from cells have been described. Two recent publications describe antisense molecules that can be used to inhibit the activity of specific miRNAs (Meister et al., 2004; Hutvagner et al., 2004), and others have proven their functionality in the heart, where they efficiently knocked-down miR-133 and miR-1 (Care et al. 2007; Yang et al. 2007). Another publication describes the use of plasmids that are transcribed by endogenous RNA polymerases and yield specific miRNAs when transfected into cells (Zeng et al., 2002). These two reagent sets have been used to evaluate single miRNAs.

### III. DELIVERY OF MIRNAS

There are a number of ways in which miRNAs can be delivered to a cell or subject. First, the miRNAs may be delivered on their own or using a formulation that promotes entry of into a cell of interest. Nanoparticles, such as lipid vehicles like liposomes, micelles, or reverse micelles, are suitable for such a purpose. A wide variety of commercial formulations are well known.

Nanoparticles are generally considered to be particulate substances having a diameter of 100 nm or less. In contrast to liposomes, which are hollow, nanoparticles tend to be solid. Thus, the drug will be less entrapped and more either embedded in or coated on the nanoparticle. Nanoparticles can be made of metals including oxides, silica, polymers such as polymethyl methacrylate, and ceramics. Similarly, nanoshells are somewhat larger and encase the delivered substances with these same materials. Either nanoparticles or nanoshells permit sustained or controlled release of the peptide or mimetic and can stabilize it to the effects of in vivo environment.

miRNAs may be delivered using expression vectors that, once introduced into cells, express the miRNA. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. The first viruses used as gene vectors were DNA viruses including the papovaviruses (simian virus 40, bovine papilloma virus, and polyoma) and adenoviruses, and RNA viruses like retroviruses. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986). One particular method for delivery involves the use of an adeno-associated virus (AAV) expression vector. Other viral vectors may be employed as expression constructs in the present disclosure, such as vaccinia virus and herpesviruses.

In order to effect expression of an miRNA, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection.

Once the expression construct has been delivered into the cell, the nucleic acid encoding the miRNA may be positioned and expressed.

In yet another embodiment of the disclosure, the expression construct may simply consist of naked recombinant DNA, RNA, or plasmids. Transfer of the construct may be performed by any of the non-viral methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA or RNA encoding a gene or transcript of interest may also be transferred in a similar manner in vivo and express the gene product.

In still another embodiment, transferring a naked RNA or modified RNA expression construct into cells may involve particle bombardment. This method depends on the ability to accelerate DNA or RNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA or lipofectamine-RNA complexes.

Liposome-mediated nucleic acid delivery and expression of foreign DNA or RNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000® is widely used and commercially available.

In certain embodiments of the disclosure, the liposome may be complexed with a hemagglutinating virus (HVJ). This has been shown to facilitate fusion with the cell membrane and promote cell entry of liposome-encapsulated nucleic acids (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA or RNA construct, it also will be desirable to include within the liposome an appropriate bacterial polymerase.

Other expression constructs which can be employed to deliver a nucleic acid encoding a particular gene or transcript into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

In a particular embodiment, the inventors contemplate the use of adeno-associated virus (AAV), a small nonpathogenic virus of the Parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short-inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for plasmid propagation and production but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genomes of most native AAVs often contain two open reading frames (ORFs), sometimes referred to as a left ORF and a right ORF. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles. In certain embodiments, the genome of an AAV particle encodes one, two or all three VP1, VP2 and VP3 polypeptides.

The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short-inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The term “recombinant,” as a modifier of vector, such as recombinant viral, e.g., lentivirus or parvovirus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′,3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild-type genome of a virus by using molecular methods to remove part of the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-218.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-218 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-218 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-218 serotype.

In certain embodiments, a method herein comprises use, administration or delivery of an rAAV1, rAAV2, rAAV3, rAAV4, rAAV5, rAAV6, rAAV7, rAAV8, rAAV9, rAAV10, rAAV11, rAAV12, rRh10, rRh74 or rAAV-218 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-218, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments and expresses the adenoviral E1a and E1b genes is used to generate recombinant AAV particles. The modified HEK293 cell line is readily transfected and provides a particularly convenient platform in which to produce rAAV particles. Methods of generating high titer AAV particles capable of transducing mammalian cells are known in the art. For example, AAV particles can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

### IV. METHODS OF TREATMENT AND ADMINISTRATION

miRNAs and vectors expressing an miRNA may, in some aspects, be administered directly to patients (in vivo) or they can be used to treat cells in vitro or ex vivo, and then administered to patients. In particular, provided herein are methods for impeding expression of the ATXN2 gene. In some of these embodiments, a human subject has SCA2 or ALS and the miRNA is delivered or produced in vivo in a therapeutically effective amount. In some embodiments, the AAV vector transduces at least about 70% of cells of the target tissue. In some embodiments, the cell is a cell of the ventricles of the brain, e.g., a motor cortex cell, brainstem cell or spinal cord cell. In other embodiments, the subject is a non-human animal (e.g., mammal) that models SCA2 or ALS.

The term “vector” refers to small carrier nucleic acid molecule, a plasmid, virus (e.g., AAV vector, retroviral vector, lentiviral vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

Any suitable cell or mammal can be administered or treated by a method or use as described herein. Typically, a mammal in need of a method as described herein is suspected of having or expressing an abnormal or aberrant ATNX2 protein that is associated with a disease state. Accordingly, as used herein, the term “therapeutic agent” refers to an miRNA that has a beneficial effect on the mammalian recipient. Thus, “therapeutic agent” embraces both therapeutic and prophylactic miRNAs.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Human subjects treated by a method or composition described herein include adults (18 years or older) and children (less than 18 years of age). Adults include the elderly. Children range in age from 1-2 years old, or from 2-4, 4-6, 6-8, 8-10, 10-12, 12-15 and 15-18 years old. Children also include infants. Infants typically range from 1-12 months of age.

In certain embodiments, a method includes administering a plurality of viral particles, such as AAV particles, to a mammal as set forth herein, where severity, frequency, progression or time of onset of one or more symptoms of a disease state, decreased, reduced, prevented, inhibited or delayed. In certain embodiments, a method includes administering a plurality of viral particles to a mammal to treat an adverse symptom of a disease state. In certain embodiments, a method includes administering a plurality of viral particles to a mammal to stabilize, delay or prevent worsening, or progression, or reverse and adverse symptom of a disease state.

In certain embodiments a method includes administering a plurality of viral particles to the central nervous system, or portion thereof as set forth herein, of a mammal and severity, frequency, progression or time of onset of one or more symptoms of a disease state, are decreased, reduced, prevented, inhibited or delayed by at least about 5 to about 10, about 10 to about 25, about 25 to about 50, or about 50 to about 100 days.

In some embodiments, a composition comprising a therapeutically effective number of virus particles containing a transgene, or containing one or more sets of different virus particles, wherein each particle in a set can contain the same type of transgene, but wherein each set of particles contains a different type of transgene than in the other sets, as described herein can be delivered.

Formulations according to the present disclosure can be used for CNS delivery via various techniques and routes including, but not limited to, intraparenchymal, intracerebral, intracerebroventricular (ICV), intrathecal (e.g., IT-Lumbar, IT-thoracic, IT-cisterna magna) administrations and any other techniques and routes for injection directly or indirectly to the CNS and/or CSF.

The present disclosure provides pharmaceutical compositions comprising an active agent and, optionally, a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil and the like. Water is a particular carrier when the pharmaceutical composition. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In general, the composition should suit the mode of administration, which can be oral, intravenous, intraarterial, intramuscular, subcutaneous or other route.

In some embodiments, a formulation is delivered to the CNS by administering into the cerebrospinal fluid (CSF) of a subject in need of treatment. In some embodiments, intrathecal administration is used to deliver viral particles into the CSF. As used herein, intrathecal administration (also referred to as intrathecal injection) refers to an injection into the spinal canal (intrathecal space surrounding the spinal cord). Various techniques may be used including, without limitation, lateral intracerebroventricular injection through a burrhole or cisternal or lumbar puncture or the like. Exemplary methods are described in Lazorthes et al. Advances in Drug Delivery Systems and Applications in Neurosurgery, 18:143-192 (1991) and Ommaya et al., Cancer Drug Delivery, 1:169-179 (1984) the contents of which are incorporated herein by reference.

According to the present disclosure, viral particles may be injected at any region surrounding the spinal canal. In some embodiments, viral particles are injected into the lumbar area or the cisterna magna or intracerebroventricularly into a cerebral ventricle space. As used herein, the term “lumbar region” or “lumbar area” refers to the area between the third and fourth or fourth and fifth lumbar (lower back) vertebrae and, more inclusively, the L2-S 1 region of the spine. Typically, intrathecal injection via the lumbar region or lumber area is also referred to as “lumbar IT delivery” or “lumbar IT administration.” The term “cisterna magna” refers to the space around and below the cerebellum via the opening between the skull and the top of the spine. Typically, intrathecal injection via cisterna magna is also referred to as “cisterna magna delivery.” The term “cerebral ventricle” refers to the cavities in the brain that are continuous with the central canal of the spinal cord. As such, intrathecal administration includes any infusion into the central canal. Typically, injections via the cerebral ventricle cavities are referred to as intracerebroventricular cerebral (ICV) delivery.

Various devices may be used for intrathecal delivery according to the present disclosure. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. In various embodiments, the fluid access port comprises a reservoir. In some embodiments, the fluid access port comprises a mechanical pump (e.g., an infusion pump). In some embodiments, an implanted catheter is connected to either a reservoir (e.g., for bolus delivery), or an infusion pump. The fluid access port may be implanted or external.

In some embodiments, intrathecal administration may be performed by either lumbar puncture (i.e., slow bolus) or via a port-catheter delivery system (i.e., infusion or bolus). In some embodiments, the catheter is inserted between the laminae of the lumbar vertebrae and the tip is threaded up the thecal space to the desired level (generally L3-L4).

A single dose volume suitable for intrathecal administration is typically small. Typically, intrathecal delivery according to the present disclosure maintains the balance of the composition of the CSF as well as the intracranial pressure of the subject. In some embodiments, intrathecal delivery is performed absent the corresponding removal of CSF from a subject. In some embodiments, a suitable single dose volume may be e.g., less than about 10 ml, 8 ml, 6 ml, 5 ml, 4 ml, 3 ml, 2 ml, 1.5 ml, 1 ml, or 0.5 ml. In some embodiments, a suitable single dose volume may be about 0.5-5 ml, 0.5-4 ml, 0.5-3 ml, 0.5-2 ml, 0.5-1 ml, 1-3 ml, 1-5 ml, 1.5-3 ml, 1-4 ml, or 0.5-1.5 ml. In some embodiments, intrathecal delivery according to the present disclosure involves a step of removing a desired amount of CSF first. In some embodiments, less than about 10 ml (e.g., less than about 9 ml, 8 ml, 7 ml, 6 ml, 5 ml, 4 ml, 3 ml, 2 ml, 1 ml) of CSF is first removed before IT administration. In those cases, a suitable single dose volume may be e.g., more than about 3 ml, 4 ml, 5 ml, 6 ml, 7 ml, 8 ml, 9 ml, 10 ml, 15 ml, or 20 ml.

Various other devices may be used to effect intrathecal administration of a therapeutic composition. For example, formulations containing desired enzymes may be given using an Ommaya reservoir which is in common use for intrathecally administering drugs for meningeal carcinomatosis (Ommaya, Lancet 2:983-84, 1963). More specifically, in this method, a ventricular tube is inserted through a hole formed in the anterior horn and is connected to an Ommaya reservoir installed under the scalp, and the reservoir is subcutaneously punctured to intrathecally deliver the particular enzyme being replaced, which is injected into the reservoir. Other devices for intrathecal administration of therapeutic compositions or formulations to an individual are described in U.S. Pat. No. 6,217,552, incorporated herein by reference. Alternatively, the viral particles may be intrathecally given, for example, by a single injection, or continuous infusion. It should be understood that the dosage treatment may be in the form of a single dose administration or multiple doses.

In one embodiment of the disclosure, the viral particles are administered by lateral cerebro-ventricular injection into the brain of a subject. The injection can be made, for example, through a burr hole made in the subject's skull. In another embodiment, the viral particles and/or other pharmaceutical formulation are administered through a surgically inserted shunt into the cerebral ventricle of a subject. For example, the injection can be made into the lateral ventricles, which are larger. In some embodiments, injection into the third and fourth smaller ventricles can also be made. In yet another embodiment, the pharmaceutical compositions used in the present disclosure are administered by injection into the cisterna magna, or lumbar area of a subject.

### V. PHARMACEUTICAL COMPOSITIONS

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering an miRNA or viral particle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the disclosure are known in the art (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

Viral particles and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles believed necessary to produce the desired effect(s). The amount necessary can be formulated in a single dose or can be formulated in multiple dosage units. The dose may be adjusted to a suitable viral particle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, and pharmaceutical compositions thereof, can be packaged in single or multiple unit dosage form for ease of administration and uniformity of dosage.

Formulations containing viral particles typically contain an effective amount, the effective amount being readily determined by one skilled in the art. The viral particles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### V1. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof.

Polynucleotides include genomic DNA, cDNA and antisense DNA, and spliced or unspliced mRNA, IRNA, tRNA and inhibitory DNA or RNA (RNAi, e.g., small or short hairpin (sh) RNA, microRNA (miRNA), small or short interfering (si) RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. A pol II promoter includes a minimal promoter that is a short DNA sequence comprised of a TATA-box and optionally other sequences that serve to specify the site of transcription initiation, to which regulatory elements are added for control of expression. A type 1 pol III promoter includes three cis-acting sequence elements downstream of the transcriptional start site: a) 5′sequence element (A block); b) an intermediate sequence element (I block); c) 3′ sequence element (C block). A type 2 pol III promoter includes two essential cis-acting sequence elements downstream of the transcription start site: a) an A box (5′ sequence element); and b) a B box (3′ sequence element). A type 3 pol III promoter includes several cis-acting promoter elements upstream of the transcription start site, such as a traditional TATA box, proximal sequence element (PSE), and a distal sequence element (DSE).

An “enhancer” is a DNA sequence that can stimulate transcription activity and may be an innate element of the promoter or a heterologous element that enhances the level or tissue specificity of expression. It is capable of operating in either orientation (5′->3′ or 3′->5′) and may be capable of functioning even when positioned either upstream or downstream of the promoter.

Enhancers may be derived in their entirety from a native gene or be composed of different elements derived from different elements found in nature, or even be comprised of synthetic DNA segments. An enhancer may comprise DNA sequences that are involved in the binding of protein factors that modulate/control effectiveness of transcription initiation in response to stimuli, physiological or developmental conditions.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, a transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence, where the promoter is capable of controlling transcription of the encoded polypeptide. A nucleic acid operably linked to an expression control element can also be referred to as an expression cassette.

In certain embodiments, an expression control element comprises a CMV enhancer.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors, to the central nervous system, such as to distinct brain regions.

A recombinant virus so modified may preferentially bind to one type of tissue (e.g., CNS tissue) over another type of tissue (e.g., liver tissue). In certain embodiments, a recombinant virus bearing a modified capsid protein may “target” brain vascular epithelia tissue by binding at level higher than a comparable, unmodified capsid protein. For example, a recombinant virus having a modified capsid protein may bind to brain ependymal tissue at a level 50% to 100% greater than an unmodified recombinant virus.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present disclosure. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type, or at least any of these values).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

By “disease” is meant any condition or disorder that damages or interferes with the normal function of a cell, tissue, or organ.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### VII. KITS

The disclosure provides kits with packaging material and one or more components therein. A kit typically includes a label or packaging insert including a description of the components or instructions for use in vitro, in vivo, or ex vivo, of the components therein. A kit can contain a collection of such components, e.g., an miRNA, recombinant vector, and/or viral particles.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### VIII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

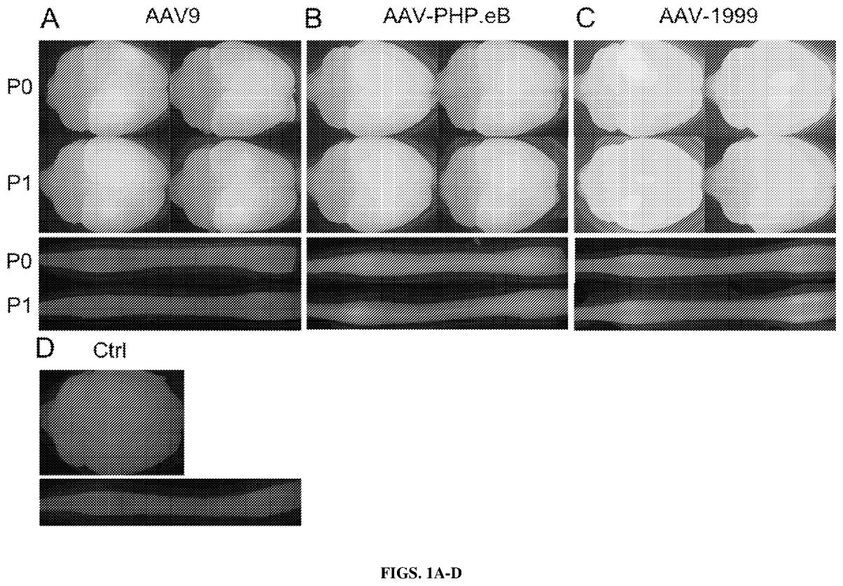

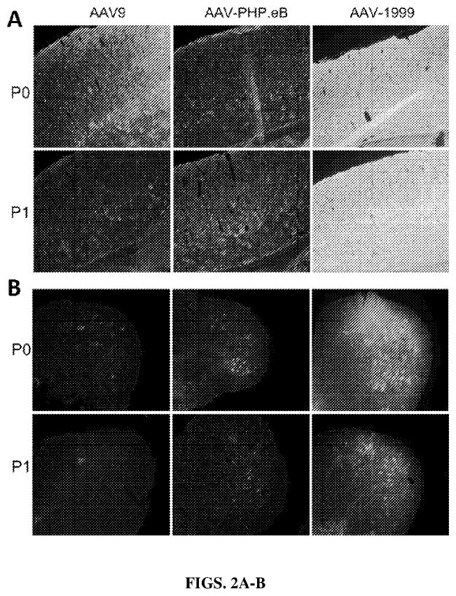

The ALS mouse model used in these studies has a rapid course of progression, requiring treatment close to birth in order to give viral vectors time to express their cargo and alter disease course. Furthermore, in ALS mice, it is necessary to transduce the upper and lower motor neurons, located in the motor cortex and the anterior horn cells of the spinal cord respectively. In order to determine the optimal vector and age of injection to achieve the desired areas and degrees of transduction, neonatal mice were injected intracranially with AAV9 variants delivering the gene encoding enhanced green fluorescent protein (eGFP) at post-natal day 0 (P0) or day 1 (P1). Compared with controls (FIG. 1D), injection with AAV9 (FIG. 1A) showed moderate cortical and weak spinal cord transduction, both of which were improved with AAV-PHP·eB delivery (FIG. 1B). Injection with AAV-1999 (FIG. 1C) showed the strongest transduction throughout both brain and spinal cord at both P0 and P1 injection times. Upon inspection of sectioned cortical (FIG. 2A) and lumbar spinal cord (FIG. 2B) tissue, AAV-1999 resulted in the strongest transduction at both P0 and P1, while the two other serotypes tested transduced more weakly and with diminished efficacy at P1.

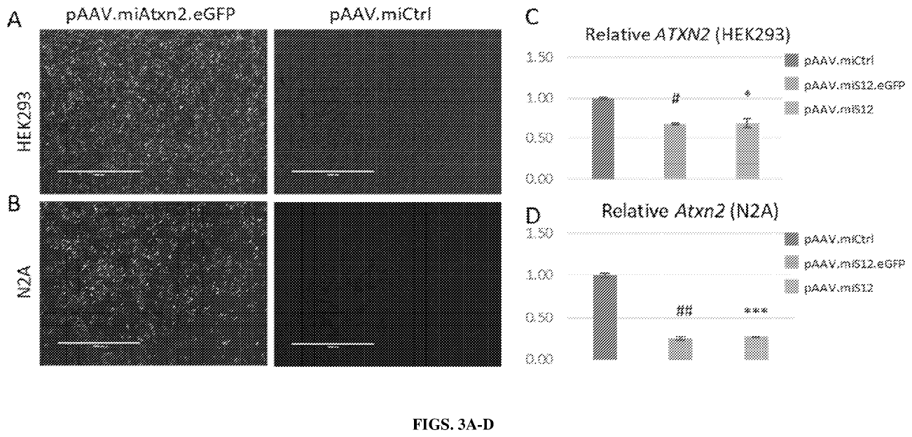

miRNA sequences targeting human and mouse Ataxin 2 mRNA (miATXN2) had previously been screened and miATXN2 miS12 selected for its targeting profile. miS12, either alone or co-expressing eGFP, was transfected into HEK293 cells (FIG. 3A, FIG. 3C) and murine N2A cells (FIG. 3B, FIG. 3D). Plasmid pAAV·miS12·eGFP showed successful transduction by fluorescence microscopy compared to a scrambled non-targeting miRNA (pAAV·miCtrl) (FIG. 3A, FIG. 3B). Both pAAV·miS12·GFP and pAAV·miS12 demonstrated knockdown of human ATXN2 by ˜35% (FIG. 3C) and mouse Atxn2 by ˜75% (FIG. 3D) as measured by QPCR.

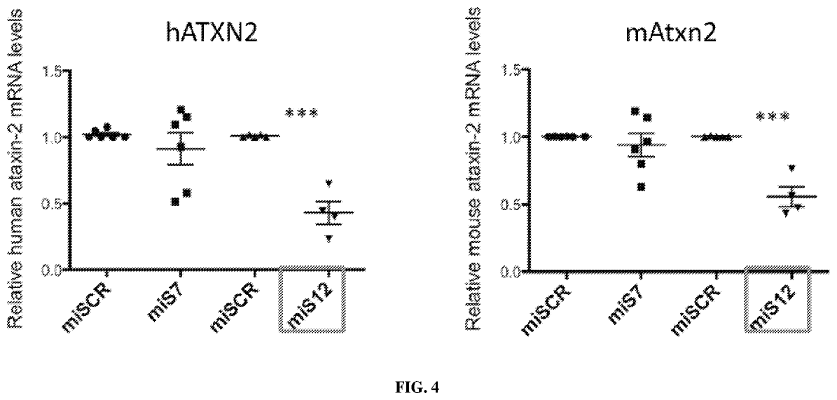

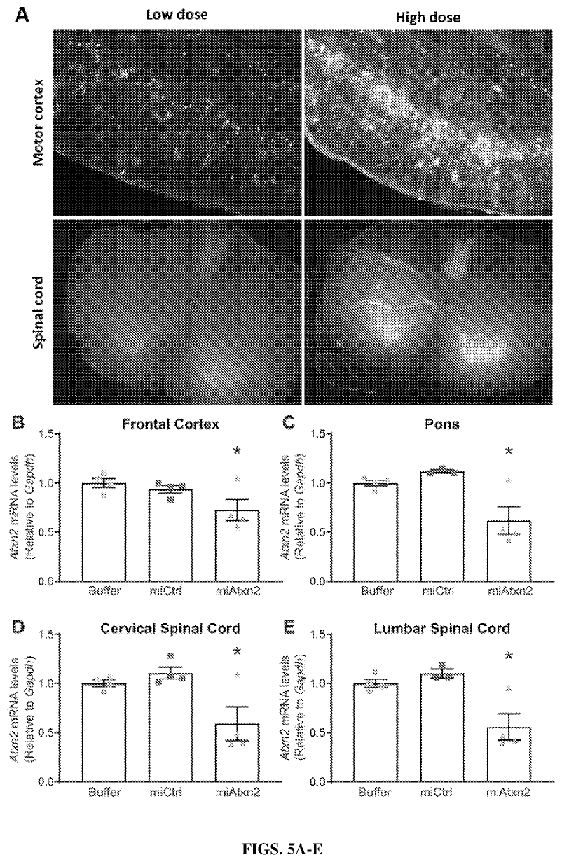

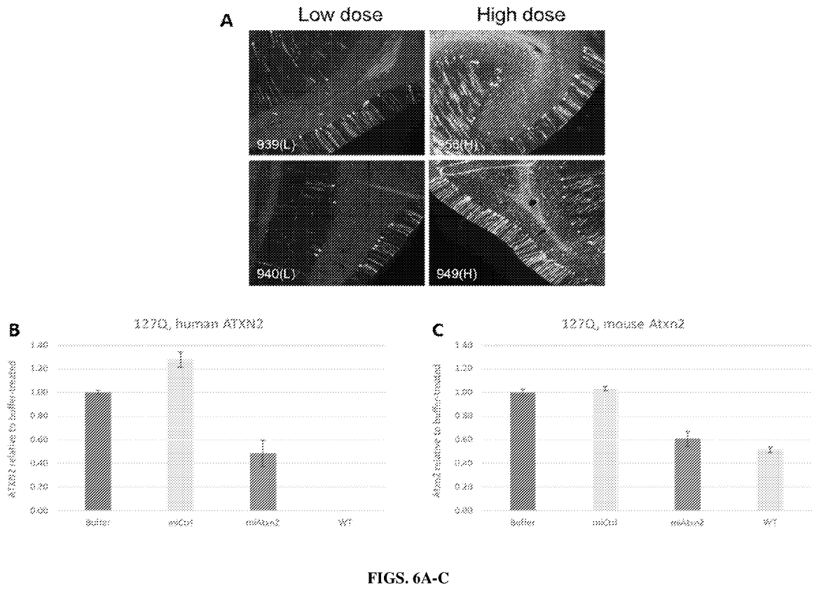

In order to test the efficacy of miS12 against both human and mouse Ataxin 2 mRNA in vivo, SCA2 mice overexpressing human ATXN2 were injected in deep cerebellar nuclei with AAV1 delivering miATXN2 variants 7 or 12 (miS7 or miS12), and human ATXN2 and mouse Atxn2 mRNA levels were measured in the cerebellum (FIG. 4). miS12 suppressed both human and mouse ATXN2/Atxn2 compared to either miCtrl or mis7. miS12 was therefore chosen for the next set of studies in vivo and delivered using AAV-PHP·eB administered systemically to adult wildtype mice. First, areas of the brain relevant to ALS were assessed. Compared to mice treated with the control vector AAV-PHP·eB-miCtrl, mice treated with AAV-PHP·eB·eGFP showed strong expression of eGFP throughout the motor cortex and anterior horn cells of the spinal cord (FIG. 5A), and mice treated with AAV-PHP·eB·miS12 showed robust knockdown of Atxn2 in the brain, brainstem, and spinal cord (FIGS. 5B-E). Next, areas of the brain relevant to SCA2 were assessed, specifically the cerebellar Purkinje cells. Compared to AAV-PHP·eB-miCtrl-treated mice, mice receiving AAV-PHP·eB·eGFP showed strong eGFP expression in cerebellar Purkinje cells (FIG. 6A), and mice treated with AAV-PHP·eB·miS12 showed robust knockdown of Atxn2 in the cerebellum (FIGS. 6B-C).

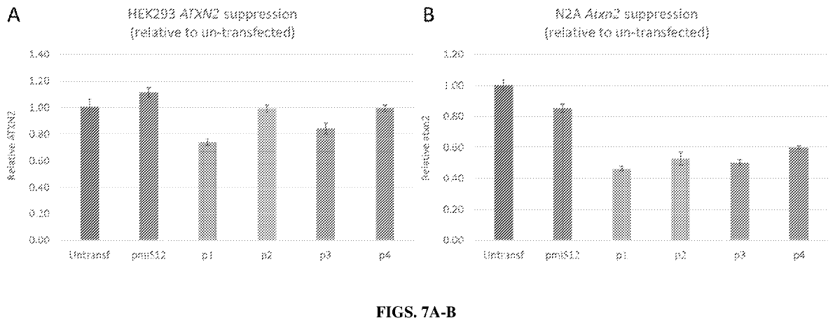

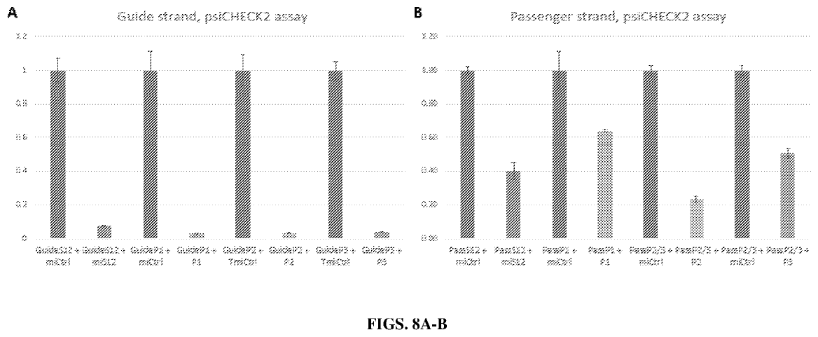

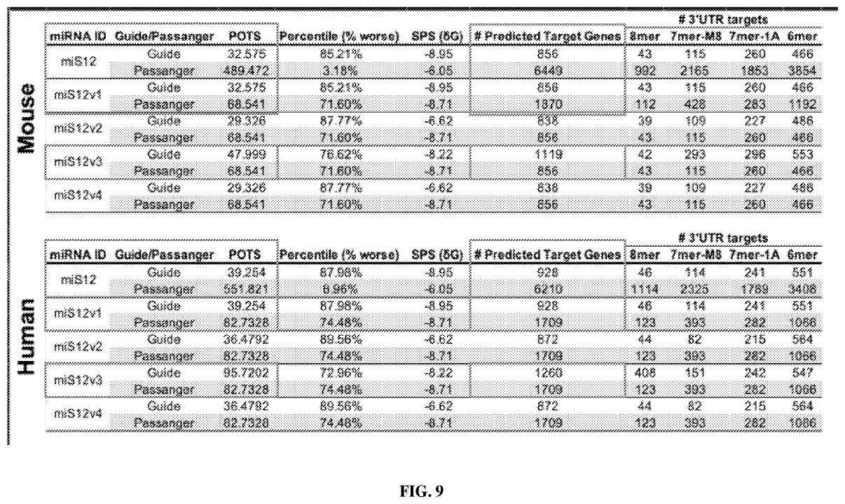

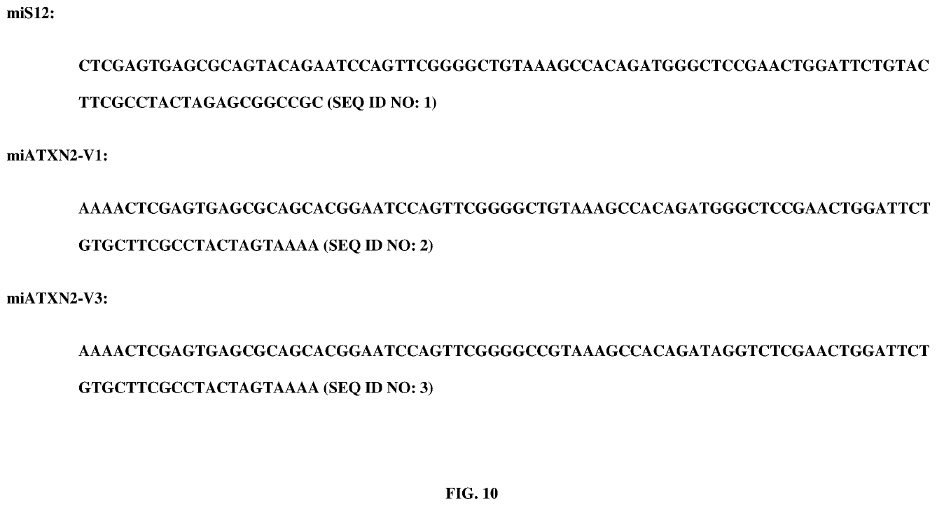

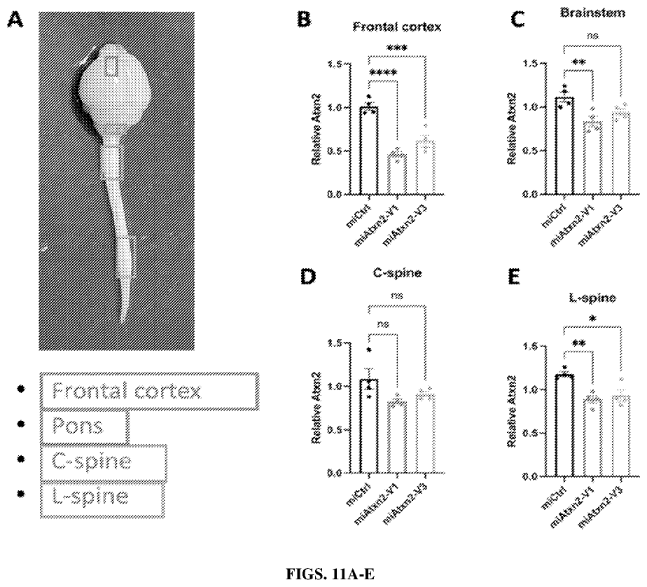

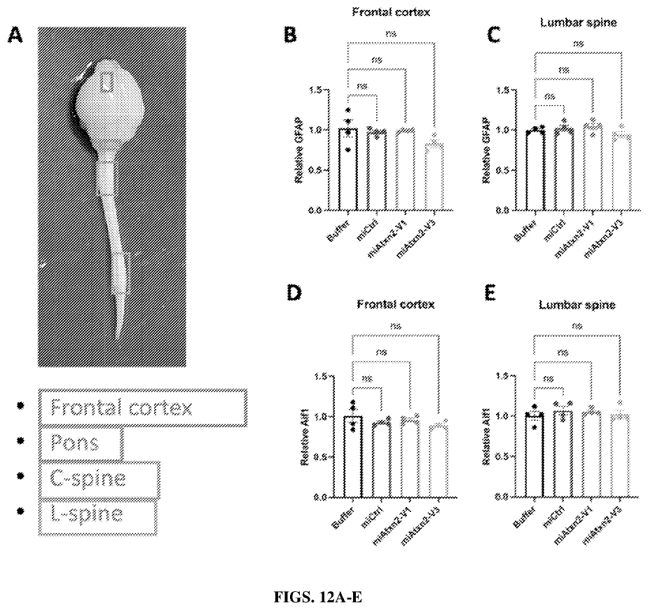

In the first efficacy study performed, it was found that the miRNA miS12 resulted in unwanted off-targeting by the passenger strand, which is supposed to be minimally active with few off-targets. Therefore, four miS12 variants were generated, referred to here and going forward as P1 (or V1 or miAtxn2-V1 or miAtxn2), P2 (or V2), P3 (or V3 or miAtxn2-V3), and P4 (or V4), in which seed-sequences and other components of the guide and passenger strands were modified to improve the targeting profiles. Knockdown of ATXN2/atxn2 RNA using miS12 was compared to that achieved by variants P1-P4 in cultured human HEK293T cells (FIG. 7A) and mouse N2A cells (FIG. 7B) using QPCR, normalizing to ATXN2/Atxn2 levels in untransfected cells. Variants P1 and P3 caused robust knockdown of both human and mouse ATXN2/atxn2 mRNA. Additional studies were performed on miS12 and the four variants: A psiCHECK-2 RNAi luciferase assay was used to compare activity and strand-loading of the guide (FIG. 8A) and passenger (FIG. 8B) strands of the miATXN2 sequences. Compared to miS12, P1 and P3 showed improved strand loading by the guide strand (FIG. 8A) and decreased strand loading by the passenger strand (FIG. 8B). In addition, miS12 and the four variants were analyzed by the program siSPOTR for predicted potential off-targeting score (POTS) and number of predicted target genes (FIG. 9). Variants V1 and V3 were chosen for subsequent experiments based on their reduced passenger-strand POTS score and off-targeting compared to miS12 (FIG. 10).

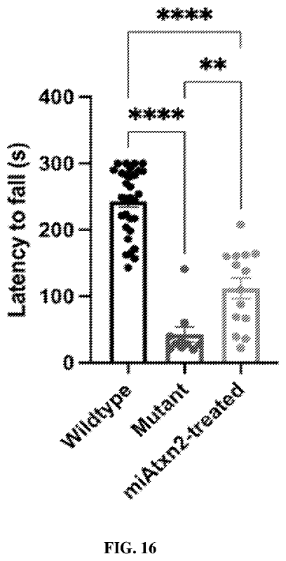

miS12, miAtxn2-V1 and miAtxn2-V3 were packaged into AAV-1999 and delivered by intracerebroventricular injection to wildtype neonatal mice at P1. The mice were of the same background strain as the TAR^{4/4} model. 3 weeks later, relevant tissues were analyzed for Atxn2 knockdown by QPCR (FIG. 11A). Robust and significant knockdown was seen in the frontal cortex (FIG. 11B) and lumbar spine (FIG. 11E) in response to treatment with either AAV·miAtxn2-V1 or AA·-miAtxn2-V3, and significant knockdown was additionally seen in the brainstem (FIG. 11C) with a trend toward knockdown in the cervical spine (FIG. 11D) in response to AAV·miAtxn2-V1 only. There was no inflammation seen in any of these regions in response to either miAtxn2 treatment, nor in response to treatment with a control, non-Atxn2-targeting miRNA (miCtrl) (FIG. 16, A-E). AAV·miAtxn2-V1 was chosen for downstream efficacy studies in the TAR^{4/4} model.

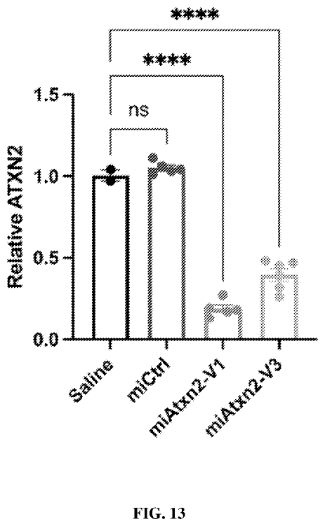

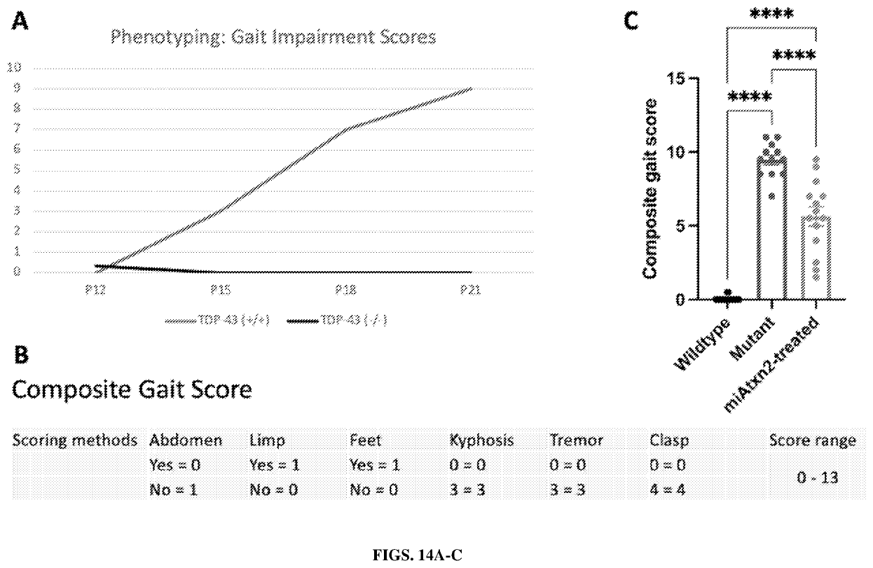

In order to assess the potential for efficacy in a SCA2 model, the inventors conducted a similar study but in wildtype mice of the same background strain as 127Q mice, again injecting at P1 at the same dose and via the same intracerebroventricular route. Four weeks after injection, the cerebellum was analyzed by QPCR. There was significant and robust knockdown in the cerebellum in response to treatment with either AAV-miAtxn2-V1 or AAV-miAtxn2-V3 (FIG. 13).

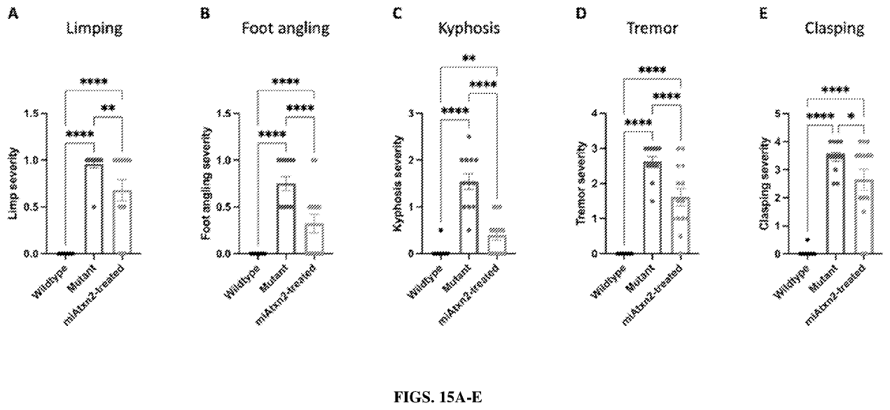

To test efficacy of these miRNAs in rescuing the ALS phenotype in the TAR^{4/4} mouse model, gait impairment scores were first calculated for TAR^{4/4} mice over time to establish a phenotype (FIG. 14A). Scores were calculated by scoring degrees of abdominal droop, limping, foot angling, kyphosis, tremor, and clasping. These scores were summed to generate a composite gait score (FIG. 14B), and it was observed that TAR^{4/4} mice show rapidly progressive gait impairment (FIG. 14A). TAR^{4/4} pups were then treated with either miATXN2-V1 or buffer and compared to wild-type littermates at postnatal day 18. miATXN2-V1-treated mice showed marked reduction in gait impairment (FIG. 14C). Each individual component of the gait impairment score was then assessed separately, and TAR^{4/4} mice showed severe impairments across measures of limping (FIG. 15A), foot angling (FIG. 15B), kyphosis (FIG. 15C), tremor (FIG. 15D), and clasping (FIG. 15E), each of which was corrected with treatment with AAV·miAtxn2-V1 (FIG. 15). Mice were also tested on Rotarod at P17, a time at which TAR^{4/4} mice showed markedly impaired performance. Treatment with AAV·miAtxn2-V1 more than doubled the mice's performance (FIG. 16).

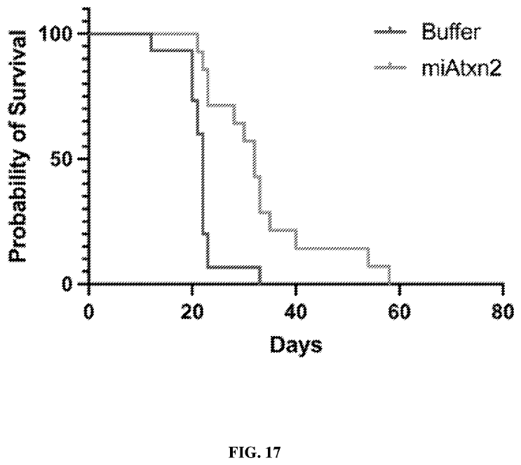

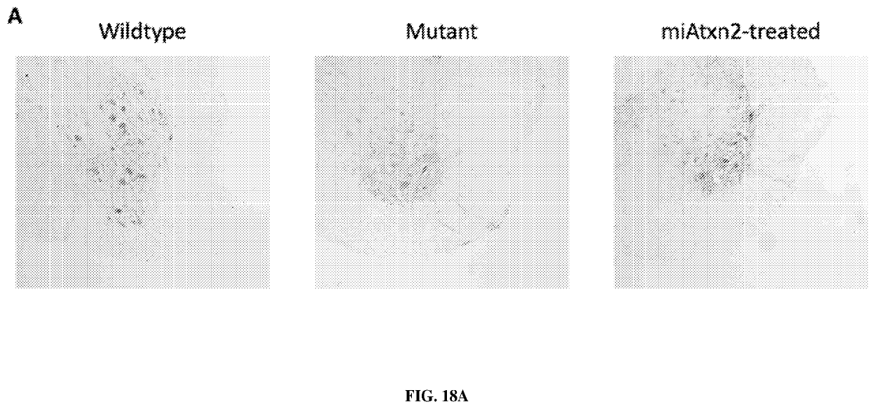

The inventors compared the survival of AAV·miAtxn2-V1-treated TAR^{4/4} mice with that of untreated TAR^{4/4} mice. Treated mice showed a 45% increase in median lifespan that was highly significant (FIG. 17).

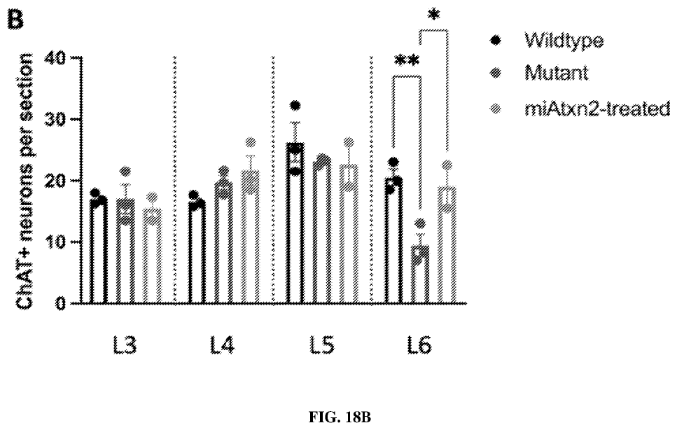

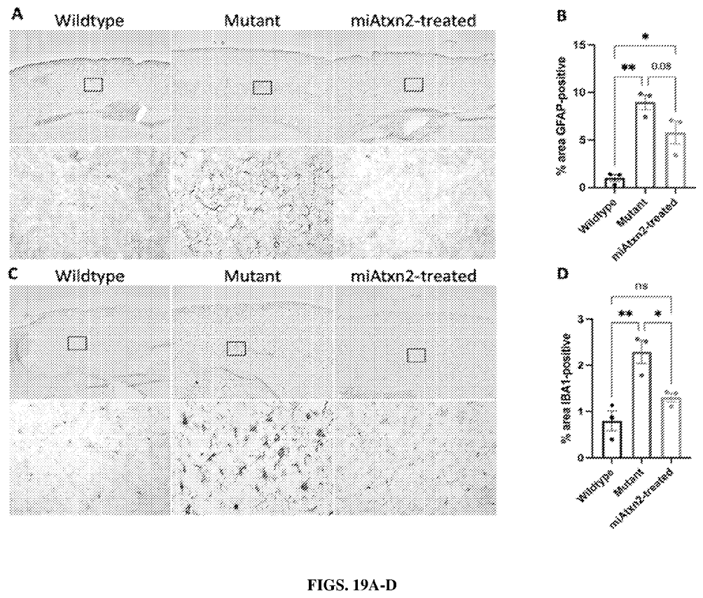

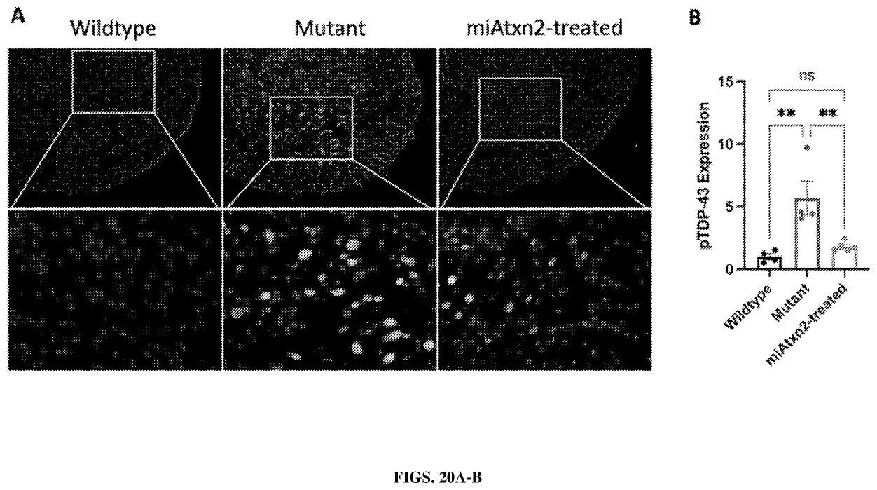

In order to more specifically determine the effects of treatment with AAV·miAtxn2-V1, treated and untreated TAR^{4/4} mice were sacrificed 21-24 days after injection, and frontal cortex and lumbar spinal cord were analyzed and compared with age-matched wildtype mice tissues. First, lower motor neurons were identified by ChAT staining (FIG. 18A) and were quantified by level. In distal regions, there was a significant increase in lower motor neuron number (FIG. 18B). Inflammation was also assessed, by measuring astrogliosis using GFAP staining and by measuring microgliosis using Iba1 staining. In the brain, there was a trend towards reduction in astrogliosis (FIGS. 19, A and B) and significant reduction in microgliosis (FIGS. 19, C and D) in mice treated with AAV·miAtxn2-V1. Finally, levels of phosphorylated TDP-43 (pTDP-43) were measured in AAV·miAtxn2-V1-treated and untreated TAR+/4 mice and compared to wildtypes. pTDP-43 is observed in the motor neurons of ALS patients. A great increase in pTDP-43 was observed in the lumbar spine in the TAR^{4/4} mouse model and was markedly reduced to wildtype levels in AAV·miAtxn2-V1-treated TAR^{4/4} mice (FIG. 20).

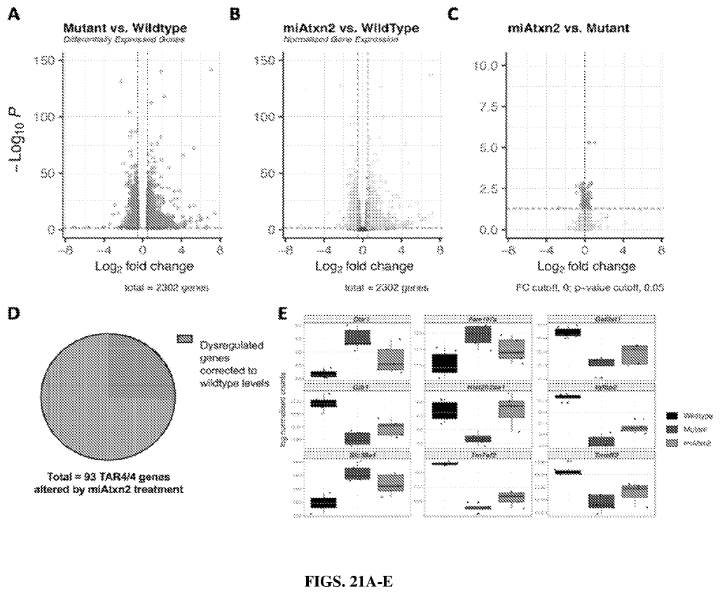

Finally, the inventors wondered about more global transcriptional changes in the TAR^{4/4} model and their degree of correction with AAV·miAtxn2-V1 treatment. Wildtype or TAR^{4/4} mice were treated at P1 with buffer and compared to TAR^{4/4} littermates treated at P1 with AAV·miATNX2-V1. The inventors measured transcriptional changes in the lumbar spinal cord harvested at postnatal day 19 and observed first that TAR44 mice showed dramatic global transcriptional changes compared to wildtypes, with 2302 genes showing at least a 0.5-fold change at cutoff adjusted p-value of <0.05 (FIG. 21A). Of those genes, 153 of them corrected when AAV·miAtxn2-V1-treated mice were compared to wildtype (FIG. 21B, blue dots). They also looked at the total genes that were significantly altered in TAR^{4/4} mice by miATXN2 treatment (FIG. 21C), of which 32 genes were corrected to their wildtype levels (FIG. 21D). Examples of 9 of those individual genes are shown FIG. 25E.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Patent Publication 20110059502
U.S. Pat. No. 6,217,552
Gonzalez et al., BMC Neurosci 10.1186/1471-2202-12-4 (2011)
Lazorthes et al. Advances in Drug Delivery Systems and Applications in Neurosurgery, 18:143-192 (1991)
Ommaya et al., Cancer Drug Delivery, 1:169-179 (1984)
Ommaya, Lancet 2:983-84 (1963)

1. A method of inhibiting expression of Ataxin 2 (ATXN2) comprising contacting a target cell with an miRNA selected from SEQ ID NO: 1, SEQ ID NO: 2, or SEQ ID NO: 3.

2. The method of claim 1, wherein the miRNA is SEQ ID NO: 1.

3. The method of claim 1, wherein the miRNA is SEQ ID NO: 2.

4. The method of claim 1, wherein the miRNA is SEQ ID NO: 3.

5. The method of claim 1, wherein the miRNA is naked miRNA.

6. The method of claim 1, wherein the miRNA is encapsulated in a nanoparticle (e.g., LNP) or liposome.

7. The method of claim 1, wherein the miRNA is encoding by an expression construct and expressed after uptake of the expression construct by the target cell.

8. The method of claim 7, wherein the expression construct is a non-viral construct.

9. The method of claim 7, wherein the expression construct is a viral construct.

10. The method of claim 9, wherein the viral construct is an AAV construct.

11. The method of claim 10, wherein the miRNA is under the control of a first promoter.

12. The method of claim 11, wherein the first promoter comprises or consists of a U6 promoter.

13. The method of claim 10, wherein the AAV comprises capsid proteins derived from or selected from the group consisting of AAV1, AAV2, AAV3, AAV4, AAV5, AAV6, AAV7, AAV8, AAV9, AAV10, AAV11, AAV12, AAV-rh74, AAV-rh10, and AAV-218 VP1, VP2 and/or VP3 capsid proteins, or capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 VP1, VP2 and/or VP3 capsid proteins.

14. The method of claim 10, wherein the AAV comprises first and second AAV ITRs derived from, comprise or consist of an AAV1, AAV2, AAV3, AAV4, AAV5, AAV6, AAV7, AAV8, AAV9, AAV10, AAV11, AAV12, AAV-rh74, AAV-rh10 or AAV-218 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 ITR sequence.

15. The method of claim 1, wherein the target cell is in a living subject.

16. The method of claim 15, wherein the administration to said living subject is systemic, regional, or localized, including intravenous delivery, or directly to the central nervous system, including but not limited to delivery to the CSF, either via intracerebroventricular delivery, intra-cisterna magna delivery, or intrathecal delivery via lumbar puncture;
intraparenchymal delivery, to the brain, brainstem, and/or spinal cord; or systemic.

17. The method of claim 1, wherein the miRNA is contacted or administered more than once.

18. The method of claim 17, wherein the miRNA is contacted or administered 2, 3, 4, 5, 6, 7, 8, 9, 10 or more times.

19. The method of claim 17, wherein the miRNA is contacted or administered monthly, every other month, every three months, every four months, every six months or annually.

20. The method of claim 15, further comprising providing an additional therapy to said subject.

21. The method of claim 9, wherein a plurality of viral particles, such as AAV vectors, are contacted or administered.

22. The method of claim 16, wherein the viral particles are administered to the living subject at a dose of about 1×10⁶ to about 1×10¹⁸ vector genomes per kilogram (vg/kg).

23. The method of claim 16, wherein the viral particles are administered to the living subject at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³ about 1×10¹⁰-1×10¹¹, about 1×10¹¹-1×10¹², about 1×10¹²-1×10¹³, or about 1×10¹³-1×10¹⁴ vg/kg of the patient.

24. The method of claim 1, wherein the living subject is human.

25. The method of claim 1, wherein the living subject is a non-human mammal.

26. The method of claim 1, wherein the human subject has been diagnosed with SCA2 or ALS.

27. The method of claim 1, wherein the human subject has been determined to be at risk of SCA2 or ALS.

28. An adeno-associated virus (AAV) encoding an miRNA selected from the group consisting of SEQ ID NO: 1, SEQ ID NO: 2, SEQ ID NO: 3, operably linked to a promoter.

29. The modified AAV of claim 28, wherein the miRNA is SEQ ID NO: 1.

30. The modified AAV of claim 28, wherein the miRNA is SEQ ID NO: 2.

31. The modified AAV of claim 28, wherein the miRNA is SEQ ID NO: 3.

32. The modified AAV of claim 28, wherein the miRNA is under the control of a first promoter.

33. The modified AAV of claim 32, wherein the first promoter comprises or consists of a U6 promoter.

34. The modified AAV of claim 29, wherein the modified AAV comprises capsid proteins derived from or selected from the group consisting of AAV1, AAV2, AAV3, AAV4, AAV5, AAV6, AAV7, AAV8, AAV9, AAV10, AAV11, AAV12, AAV-rh74, AAV-rh10, and AAV-218 VP1, VP2 and/or VP3 capsid proteins, or capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins.

35. The modified AAV of claim 28, wherein the modified AAV comprises first and second AAV ITRs derived from, comprise or consist of an AAV1, AAV2, AAV3, AAV4, AAV5, AAV6, AAV7, AAV8, AAV9, AAV10, AAV11, AAV12, AAV-rh74, AAV-rh10 or AAV-218 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 ITR sequence.

36. A pharmaceutical composition comprising AAV of claim 28 and a pharmaceutically acceptable carrier.

37. An isolated and purified nucleic acid comprising a sequence selected from the group consisting of SEQ ID NO: 1, SEQ ID NO: 2, or SEQ ID NO: 3, or a sequence having at least about 90% sequence identity therewith.

38. The nucleic acid of claim 37, wherein the sequence is SEQ ID NO: 1 or a sequence having at least about 92%, 95%, 96%, 97%, 98% or 99% sequence identity therewith.

39. The nucleic acid of claim 37, wherein the sequence is SEQ ID NO: 2 or a sequence having at least about 92%, 95%, 96%, 97%, 98% or 99% sequence identity therewith.

40. The nucleic acid of claim 37, wherein the sequence is SEQ ID NO: 3 or a sequence having at least about 92%, 95%, 96%, 97%, 98% or 99% sequence identity therewith.

41. The nucleic acid of claim 37, wherein the sequence is located in a replicable vector.

42. The nucleic acid of claim 41, wherein the replicable vector is a non-viral vector.

43. The nucleic acid of claim 41, wherein the replicable vector is a viral vector.

44. The nucleic acid of claim 43, wherein the viral vector is an AAV vector.

45. The nucleic acid of claim 44, wherein the miRNA is under the control of a first promoter.

46. The nucleic acid of claim 45, wherein the first promoter comprises or consists of a U6 promoter.

47. The nucleic acid of claim 44, wherein the modified AAV comprises capsid proteins derived from or selected from the group consisting of AAV1, AAV2, AAV3, AAV4, AAV5, AAV6, AAV7, AAV8, AAV9, AAV10, AAV11, AAV12, AAV-rh74, AAV-rh10, and AAV-218 VP1, VP2 and/or VP3 capsid proteins, or capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 VP1, VP2 and/or VP3 capsid proteins.

48. The nucleic acid of claim 44, wherein the modified AAV comprises first and second AAV ITRs derived from, comprise or consist of an AAV1, AAV2, AAV3, AAV4, AAV5, AAV6, AAV7, AAV8, AAV9, AAV10, AAV11, AAV12, AAV-rh74, AAV-rh10 or AAV-218 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-218 ITR sequence.


SEQUENCE LISTING (8 sequences)

SEQ ID NO: 1 - RNA, 94 nt, synthetic construct
  source 1..94: mol_type = other RNA
ctcgagtgagcgcagtacagaatccagttcggggctgtaaagccacagatgggctccgaactggattctgtacttcgcctactagagcggccgc

SEQ ID NO: 2 - RNA, 94 nt, synthetic construct
  source 1..94: mol_type = other RNA
aaaactcgagtgagcgcagcacggaatccagttcggggctgtaaagccacagatgggctccgaactggattctgtgcttcgcctactagtaaaa

SEQ ID NO: 3 - RNA, 94 nt, synthetic construct
  source 1..94: mol_type = other RNA
aaaactcgagtgagcgcagcacggaatccagttcggggccgtaaagccacagataggtctcgaactggattctgtgcttcgcctactagtaaaa

SEQ ID NO: 4 - RNA, 4341 nt, Homo sapiens
  source 1..4341: mol_type = mRNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 5 - RNA, 3993 nt, Homo sapiens
  source 1..3993: mol_type = mRNA
cccgagaaagcaacccagcgcgccgcccgctcctcacgtgtccctcccggccccggggccacctcacgttctgcttccgtctgacccctccgacttccgaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcagtgcgccagtgaggctctggcaaggtgtgggctagagatgcgactcagttggatctatctctcagaaggctaccttgctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 6 - RNA, 3755 nt, Homo sapiens
  source 1..3755: mol_type = mRNA
cccgagaaagcaacccagcgcgccgcccgctcctcacgtgtccctcccggccccggggccacctcacgttctgcttccgtctgacccctccgacttccgatttcttttgatggaatctatgcaaatatgaggatggttcatatacttacatcagttgtttgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgtttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 7 - RNA, 4280 nt, Homo sapiens
  source 1..4280: mol_type = mRNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaataccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 8 - RNA, 4347 nt, Homo sapiens
  source 1..4347: mol_type = mRNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcaggtaaagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga